(19) United States
(12) Patent Application Publication
(10) Pub. No.: US 2017/0231894 A1
(43) Pub. Date: Aug. 17, 2017

(54) NON-TACKY GEL-TYPE COSMETIC COMPOSITION WITH IMPROVED WEAR PROPERTY

(71) Applicant: L'OREAL, Paris (FR)

(72) Inventors: Laure DAUBERSIES, Paris (FR); Philippe ILEKTI, Maisons-Alfort (FR)

(73) Assignee: L'OREAL, Paris (FR)

(21) Appl. No.: 15/507,319

(22) PCT Filed: Aug. 26, 2015
(86) PCT No.: PCT/IB2015/056469

§ 371 Date: Feb. 28, 2017

(30) Foreign Application Priority Data
Aug. 28, 2014 (FR) 14 58058

(51) Int. Cl. A61K 8/88; A61K 8/86; A61K 8/25; A61K 8/81; A61Q 1/10; A61K 8/04

(52) U.S. Cl. CPC A61K 8/88; A61Q 1/10; A61K 8/042; A61K 8/25; A61K 8/8117; A61K 8/86; A61K 2800/882; A61K 2800/54; A61K 2800/594

(57) ABSTRACT

The present invention is directed towards a composition, especially a cosmetic composition,in particular for coatingkeratin materials, especially keratin fibres, more particularlythe eyelashes, comprising: -at least one aqueous phase gelled with at least one hydrophilic gelling agent chosen from synthetic polymeric gelling agents, mixed silicates, and mixtures thereof; and at least one oily phase gelled with at least one lipophilic gelling agent chosen from polymeric gelling agents, particulate gelling agents, and mixtures thereof, said oily phase also comprising at least one volatile oil; said phases forming therein a macroscopically homogeneous mixture; said composition also comprising at least one tackifying resin, said composition comprising a water content at least equal to 15% by weight, relative to the total weight of the composition, said composition comprising from 10% to 70% by weight of volatile oil(s) relative to the total weight of said compositions.

The present invention relates to the field of caring for and/or making up keratin materials, especially the skin, the lips and/or keratin fibres, and is more particularly directed towards proposing compositions, especially cosmetic compositions, with improved wear property over time while at the same time limiting the undesired phenomenon of transient tack.

The term “keratin materials” preferably means human keratin materials, especially the skin, the lips and/or keratin fibres.

The present invention proves to be most particularly advantageous for caring for and/or making up keratin fibres.

The term “keratin fibres” especially means the eyelashes, eyebrows, bodily hair and/or head hair, in particular the eyelashes and/or the eyebrows, and preferably the eyelashes.

One of the problems generally encountered during the coating of keratin fibres, for example the eyelashes, is that the film obtained has a tendency to crumble away over time. Grains become detached from the applied film and become deposited in the region of said keratin fibres, leaving, in the case of mascaras, unaesthetic traces especially around the eyes.

An additional problem is that the film thus embrittled has reduced resistance to friction, especially on contact with the fingers or even with water, for example when bathing or taking a shower, or alternatively under hot and humid climatic conditions. The coating is then no longer sufficiently resistant and shows poor wear property over time.

To solve this problem, it is already known practice to use tackifying compounds which have the effect, by virtue of their tackifying properties, of improving the wear property properties of compositions containing them.

Nevertheless, this tackifying nature has the drawback of making keratin fibres stick together, this problem being exacerbated especially when the composition comprises a volatile oil which thus evaporates after application. For obvious reasons, this is unacceptable to the user.

There thus remains a need for compositions that are capable of forming a deposited film that has good wear property over time and good resistance to rubbing, without having the abovementioned drawbacks, and in particular showing significantly reduced tackiness when compared with that presented by the conventional formulations described above.

Contrary to all expectation, the inventors have found that the presence of a tackifying resin in a particular architecture in terms of galenical formulation makes it possible precisely satisfy this expectation.

Thus, according to a first of its aspects, the present invention relates to a composition, especially a cosmetic composition, in particular for coating keratin materials, especially keratin fibres, more particularly the eyelashes, comprising:

at least one aqueous phase gelled with at least one hydrophilic gelling agent chosen from synthetic polymeric gelling agents, mixed silicates, and mixtures thereof, and

at least one oily phase gelled with at least one lipophilic gelling agent chosen from polymeric gelling agents, particulate gelling agents, and mixtures thereof, said oily phase also comprising at least one volatile oil;

said phases forming therein a macroscopically homogeneous mixture;

said composition also comprising at least one tackifying resin,

said composition comprising a water content at least equal to 15% by weight, relative to the total weight of the composition,

said composition comprising from 10% to 70% by weight of volatile oil(s) relative to the total weight of said composition.

Thus, according to a preferred embodiment, the present invention relates to a composition, especially a cosmetic composition, in particular for coating keratin fibres, more particularly the eyelashes, comprising:

at least one aqueous phase gelled with at least one hydrophilic gelling agent chosen from synthetic polymeric gelling agents, mixed silicates, and mixtures thereof; and

at least one oily phase gelled with at least one lipophilic gelling agent chosen from polymeric gelling agents, particulate gelling agents, and mixtures thereof; said oily phase also comprising at least one volatile oil and at least one tackifying resin,

said phases forming therein a macroscopically homogeneous mixture,

preferably

said composition comprising a water content at least equal to 15% by weight, relative to the total weight of the composition

said composition comprising from 10% to 70% by weight of volatile oil(s) relative to the total weight of said composition.

Contrary to all expectation, the inventors have in fact found that the presence of a tackifying resin in a galenical architecture that is in the form of a macroscopically homogeneous mixture of a gelled aqueous phase and of a gelled oily phase as defined above gives access to a mascara formulation that has the expected properties in terms of wear property due to the tackifying resin, but which can advantageously give a fine, transfer-free deposit that has reduced transient tack by virtue of the gelled aqueous phase.

The compositions according to the invention may especially be makeup compositions intended for affording solely by their use on the eyelashes the desired makeup effect, but may also be non-pigmented or coloured compositions intended either to be superposed on a makeup already deposited on the eyelashes or coated with an associated film of makeup, and they are then termed, respectively, top coat or base coat. They may also be compositions intended to afford only care on keratin fibres and in particular the eyelashes.

Certainly, “gel-gel” compositions have already been proposed in the cosmetics field. Formulations of this type combine a gelled aqueous phase with a gelled oily phase. Thus, gel/gel formulations are described in Almeida et al., Pharmaceutical Development and Technology, 2008, 13:487, tables 1 and 2, page 488; WO 99/65455; PI 0405758-9; WO 99/62497; JP 2005-112834 and WO 2008/081175. However, to the inventors' knowledge, this type of formulation has never been proposed for purposes of affording cosmetic compositions that are especially intended for makeup and/or care, in particular for coating keratin fibres, and which combine the advantages of formulations comprising a tackifying resin, i.e. excellent wear property over time, while at the same time being free of the drawbacks usually associated.

According to another of its aspects, a subject of the invention is also a process for preparing a composition, especially a cosmetic composition, in particular for coating keratin materials, preferably keratin fibres such as the eyelashes, comprising at least one step of mixing:

an aqueous phase gelled with at least one hydrophilic gelling agent chosen from synthetic polymeric gelling agents, mixed silicates, and mixtures thereof; and

at least one oily phase gelled with at least one lipophilic gelling agent chosen from polymeric gelling agents, particulate gelling agents, and mixtures thereof, said oily phase also comprising at least one volatile oil;

under conditions suitable for obtaining a macroscopically homogeneous mixture;

said composition also comprising at least one tackifying resin, preferably

said composition comprising a water content at least equal to 15% by weight, relative to the total weight of the composition,

said composition comprising from 10% to 70% by weight of volatile oil(s) relative to the total weight of said composition.

According to one embodiment variant, this process may advantageously comprise a step of mixing at least three or even more gelled phases.

For obvious reasons, the number of gelled aqueous phases and of gelled oily phases to be considered for forming a composition according to the invention may range for each of the two types of phase beyond two.

Advantageously, the mixing of the phases may be performed at room temperature.

However, the process of the invention may comprise, if necessary, a step of heating the mixture.

According to one embodiment variant, the final formulation may be manufactured without following a particular order of introduction of the various constituents and, in certain cases, a “one-pot” manufacture may be performed.

According to a particular embodiment, the representative gelled phases of the same type of architecture are gelled with a different gelling agent.

Multi-phase formulas may thus be developed.

According to another of its aspects, a subject of the invention is also a process, especially a cosmetic process, for making up and/or caring for keratin materials, in particular keratin fibres, especially the eyelashes, comprising at least one step which consists in applying to said keratin materials a composition in accordance with the invention.

According to yet another of its aspects, the present invention relates to a process, especially a cosmetic process, for making up and/or caring for keratin materials, in in particular keratin fibres, especially the eyelashes, comprising at least the application to said keratin materials of a macroscopically homogeneous composition obtained by extemporaneous mixing, before application or at the time of application to said keratin materials, of at least one aqueous phase gelled with at least one hydrophilic gelling agent chosen from synthetic polymeric gelling agents, mixed silicates, and mixtures thereof, and at least one oily phase gelled with at least one lipophilic gelling agent chosen from polymeric gelling agents, particulate gelling agents, and mixtures thereof, said oily phase also comprising at least one volatile oil; said composition also comprising at least one tackifying resin.

Cosmetic Composition

To begin with, it is important to note that a composition according to the invention is different from an emulsion.

An emulsion generally consists of an oily liquid phase and an aqueous liquid phase. It is a dispersion of droplets of one of the two liquid phases in the other. The size of the droplets forming the dispersed phase of the emulsion is typically about a micrometre (0.1 to 100 μm). Furthermore, an emulsion requires the presence of a surfactant or of an emulsifier to ensure its stability over time.

In contrast, a composition according to the invention consists of a macroscopically homogeneous mixture of two immiscible gelled phases. These two phases both have a gel-type texture. This texture is especially reflected visually by a consistent and/or creamy appearance.

The term “macroscopically homogeneous mixture” means a mixture in which each of the gelled phases cannot be individualized by the naked eye. More precisely, in a composition according to the invention, the gelled aqueous phase and the gelled oily phase interpenetrate and thus form a stable, consistent product. This consistency is achieved by mixing interpenetrated macrodomains. Thus, by microscope, the composition according to the invention is very different from an emulsion. A composition according to the invention cannot be characterized either as having a “sense”, i.e. an O/W or W/O sense, this means that a continuous phrase and a dispersed phase cannot be defined.

Thus, a composition according to the invention has a consistency of gel type. The stability of the composition is long-lasting without surfactant. Consequently, a composition, especially a cosmetic composition according to the invention, does not require any surfactant or silicone emulsifier to ensure its stability over time.

A composition according to the invention is distinguishable from an emulsion by mean of at least one of the following tests : test using a dyestuff, drop test and dilution test.

Test using a Dyestuff

It is known practice from the prior art to observe the intrinsic nature of a mixture of aqueous and oily gels in a gel-type composition, for example, by introducing a dyestuff either into the aqueous gelled phase or into the lipophilic gelled phase, before the formation of the gel-type composition. During visual inspection, in a gel-type composition, the dyestuff appears uniformly dispersed, even if the dye is present solely in the gelled aqueous phase or in the gelled oily phase. Specifically, if two different dyes of different colours are introduced, respectively, into the oily phase and into the aqueous phase, before formation of the gel-type composition, the two colours may be observed as being uniformly dispersed throughout the gel-type composition. This is different from an emulsion in which, if a dye, which is soluble in water or soluble in oil, is introduced, respectively, into the aqueous and oily phases, before forming the emulsion, the colour of the dye present will only be observed in the outer phase (Remington: The Science and Practice of Pharmacy, 19th Edition (1995), Chapter 21, page 282).

Drop Test

It is also known practice to distinguish a gel-type composition from an emulsion by performing a “drop test”. This test consists in demonstrating the bi-continuous nature of a gel-type composition. Specifically, as mentioned previously, the consistency of a composition is obtained by means of the interpenetration of the aqueous and oily gelled domains. Consequently, the bi-continuous nature of a gel-type composition may be demonstrated by means of a simple test with, respectively, hydrophilic and hydrophobic solvents. This test consists in depositing, firstly, one drop of a hydrophilic solvent on a first sample of the test composition, and, secondly, one drop of a hydrophobic solvent on a second sample of the same test composition, and in analysing the behaviour of the two drops of solvents. In the case of an O/W emulsion, the drop of hydrophilic solvent diffuses into the sample and the drop of hydrophobic solvent remains at the surface of the sample. In the case of a W/O emulsion, the drop of hydrophilic solvent remains at the surface of the sample and the drop of hydrophobic solvent diffuses throughout the sample. Finally, in the case of a gel-type composition (bi-continuous system), the hydrophilic and hydrophobic drops diffuse throughout the sample.

Dilution Test

In the case of the present invention, the test that will be preferred for distinguishing a gel-type composition from an emulsion is a dilution test. Specifically, in a gel-type composition, the aqueous and oily gelled domains interpenetrate and form a consistent and stable composition, in which the behaviour in water and in oil is different from the behaviour of an emulsion. Consequently, the behaviour during dilution of a gel-type composition (bi-continuous system) may be compared to that of an emulsion, obviously the behaviour during dilution of a gel/gel-type composition and the one of a emulsion will be different.

More specifically, the dilution test consists in placing 40 g of product and 160 g of dilution solvent (water or oil) in a 500 mL plastic beaker. The dilution is performed with controlled stirring to avoid any emulsification. In particular, this is performed using a planetary mixer: Speed Mixer TM DAC400FVZ. The speed of the mixer is set at 1500 rpm for 4 minutes. Finally, observation of the resulting sample is performed using an optical microscope at a magnification of ×100 (×10×10). It may be noted that oils such as Parleam® and Xiameter PMX-200 Silicone Fluid 5CS® sold by Dow Corning are suitable as dilution solvent, in the same respect as one of the oils contained in the composition.

In the case of a gel-type composition (bi-continuous system), when it is diluted in oil or in water, a heterogeneous appearance is always observed. When a gel-type composition (bi-continuous system) is diluted in water, pieces of oily gel in suspension are observed, and when a gel-type composition (bi-continuous system) is diluted in oil, pieces of aqueous gel in suspension are observed.

In contrast, during dilution, emulsions have a different behaviour. When an O/W emulsion is diluted in an aqueous solvent, it gradually reduces without having a heterogeneous and lumpy appearance. This same O/W emulsion, on dilution with oil, has a heterogeneous appearance (pieces of O/W emulsion suspended in the oil). When a W/O emulsion is diluted with an aqueous solvent, it has a heterogeneous appearance (pieces of W/O emulsion suspended in the water). This same W/O emulsion, when diluted in oil, gradually reduces without having a heterogeneous and lumpy appearance.

In a preferred embodiment, the composition comprises less than 5% surfactant, better still less than 2%, or even less than 1% and free from surfactant.

According to the present invention, the aqueous gelled phase and the oily gelled phase forming a composition according to the invention are present therein in a weight ratio ranging from 10/90 to 90/10. More preferentially, the aqueous phase and the oily phase are present in a weight ratio ranging from 30/70 to 70/30.

The ratio between the two gelled phases is adjusted according to the desired cosmetic properties.

Advantageously, a composition according to the invention may thus be in the form of a creamy gel with a minimum stress below which it does not flow unless it has been subjected to an external mechanical stress.

As emerges from the text hereinbelow, a composition according to the invention may have a minimum threshold stress of 1.5 Pa and in particular greater than 10 Pa.

The composition according to the invention may have a maximum threshold stress of 10 000 Pa.

It also advantageously has a stiffness modulus G* at least equal to 400 Pa and preferably greater than 1000 Pa. The composition according to the invention may have a stiffness modulus G* preferably lower than 50 000 Pa.

The ratio of the hydrophilic phase viscosity/lipophilic phase viscosity (measured at 25° C. and 100 s⁻¹) preferably ranges from 0.5 and 1.5 preferably from 0.2 and 3.

According to an advantageous embodiment variant, the gelled phases under consideration to form a composition according to the invention have, respectively, a threshold stress of greater than 1.5 Pa and preferably greater than 10 Pa.

The gelled phases under consideration to form a composition according to the invention may have a threshold stress lower than 10 000 Pa.

Characterization of the threshold stresses is performed by oscillating rheology measurements. Methodology is proposed in the illustrative chapter of the present text.

In general, the corresponding measurements are taken at 25° C. using a Haake RS600 imposed-stress rheometer equipped with a plate-plate measuring body (60 mm diameter) fitted with an anti-evaporation device (bell jar). For each measurement, the sample is placed delicately in position and the measurements start 5 minutes after placing the sample in the jaws (2 mm). The test composition is then subjected to a stress ramp from 10⁻² to 10³ Pa at a set frequency of 1 Hz.

A composition according to the invention may also have a certain consistency. This consistency may be characterized by a stiffness modulus G* which, under this minimum stress threshold, may be at least equal to 400 Pa and preferably greater than 1000 Pa. The value G* of a composition may be obtained by subjecting the composition under consideration to a stress ramp from 10⁻² to 10³ Pa at a set frequency of 1 Hz.

A composition according to the invention has a viscosity preferentially ranging from 0.5 to 50 Pa·s, measured at room temperature of 25° C. using a Rheomat RM100® rheometer.

Dry Extract

The composition according to the invention advantageously comprises a solids content of greater than or equal to 25%, preferably 30%, better still 35%, in particular 40%, or even 42% and preferentially 45%.

The composition according to the invention advantageously comprises a solids content ranging from 20 to 60%, preferably from 25 to 55% more preferably from 30 to 50% relative to the weight of the composition.

The aqueous phase of the composition according to the invention advantageously comprises water in an amount ranging from 80 to 95% relative to the weight of the aqueous phase.

The composition according to the invention advantageously comprises water in an amount ranging from 30 to 70% relative to the weight of the composition.

The oily phase of the composition according to the invention advantageously comprises oil(s) in an amount ranging from 40 to 70% relative to the weight of the oily phase.

For the purposes of the present invention, the “solids content” denotes the content of non-volatile material.

The amount of dry extract (abbreviated as DE) of a composition according to the invention is measured using a commercial halogen desiccator (Halogen Moisture Analyzer HR 73) from Mettler Toledo. The measurement is taken on the basis of the weight loss of a sample dried by halogen heating and thus represents the percentage of residual material once the water and the volatile materials have evaporated off.

This technique is fully described in the machine documentation provided by Mettler Toledo.

The measuring protocol is as follows:

About 2 g of composition, referred to hereinbelow as the sample, are spread out on a metal crucible, which is introduced into the halogen desiccator mentioned above. The sample is then subjected to a temperature of 105° C. until a constant weight is obtained. The wet mass of the sample, corresponding to its initial mass, and the dry mass of the sample, corresponding to its mass after halogen heating, are measured by means of a precision balance.

The experimental error associated with the measurement is of the order of ±2%.

The solids content is calculated in the following manner:

Solids content (expressed as wight%)=100 ×(dry weight/wet weight)

Hydrophilic Gelling Agent

For the purposes of the present invention, the term “hydrophilic gelling agent” means a compound that is capable of gelling the aqueous phase of the compositions according to the invention.

The gelling agent is hydrophilic and is thus present in the aqueous phase of the composition.

The gelling agent may be water-soluble or water-dispersible.

As stated above, the aqueous phase of a composition according to the invention is gelled with at least one hydrophilic gelling agent chosen from synthetic polymeric gelling agents, mixed silicates and mixtures thereof.

Preferably, the hydrophilic gelling agent may be chosen from synthetic polymeric gelling agents.

I. Synthetic polymeric Gelling Agents

For the purposes of the invention, the term “synthetic” means that the polymer is neither naturally existing nor a derivative of a polymer of natural origin.

The synthetic polymeric hydrophilic gelling agent under consideration according to the invention may or may not be particulate.

For the purposes of the invention, the term “particulate” means that the polymer is in the form of particles, preferably spherical particles.

As emerges from the text hereinbelow, the polymeric hydrophilic gelling agent is advantageously chosen from crosslinked acrylic homopolymers or copolymers; associative polymers, in particular associative polymers of polyurethane type; polyacrylamides and crosslinked and/or neutralized 2-acrylamido-2-methylpropanesulfonic acid polymers and copolymers; modified or unmodified carboxyvinyl polymers, and mixtures thereof, especially as defined below.

Synthetic polymeric gelling agents may be detailed under the following subfamilies:

1. Associative polymers,

2. Polyacrylamides and crosslinked and/or neutralized 2-acrylamido-2-methylpropanesulfonic acid polymers and copolymers, and

3. Modified or unmodified carboxyvinyl polymers.

1 Associative Polymers

For the purposes of the present invention, the term “associative polymer” means any amphiphilic polymer comprising in its structure at least one fatty chain and at least one hydrophilic portion. The associative polymers in accordance with the present invention may be anionic, cationic, nonionic or amphoteric.

Associative Anionic Polymers

Among the associative anionic polymers that may be mentioned are those comprising at least one hydrophilic unit, and at least one fatty-chain allyl ether unit, more particularly those whose hydrophilic unit is formed by an unsaturated ethylenic anionic monomer, more particularly by a vinylcarboxylic acid and most particularly by an acrylic acid or a methacrylic acid or mixtures thereof, and whose fatty-chain allyl ether unit corresponds to the monomer of formula (I) below:

CH₂═C(R′)CH₂OBₙR (I)

in which R′ denotes H or CH₃, B denotes the ethylenoxy radical, n is zero or denotes an integer ranging from 1 to 100, R denotes a hydrocarbon-based radical chosen from alkyl, arylalkyl, aryl, alkylaryl and cycloalkyl radicals, comprising from 8 to 30 carbon atoms, preferably from 10 to 24 and even more particularly from 12 to 18 carbon atoms.

Anionic amphiphilic polymers of this type are described and prepared, according to an emulsion polymerization process, in patent EP 0 216 479.

Among the associative anionic polymers that may also be mentioned are maleic anhydride/C₃₀-C₃₈-α-olefin/alkyl maleate terpolymers, such as the product maleic anhydride/C₃₀-C₃₈-α-olefin/isopropyl maleate copolymer sold under the name Performa V 1608 by the company New Phase Technologies.

Among the associative anionic polymers, mention may be made, according to a preferred embodiment, of copolymers comprising among their monomers an α,β-monoethylenically unsaturated carboxylic acid and an ester of an α,β-monoethylenically unsaturated carboxylic acid and of an oxyalkylenated fatty alcohol.

Preferentially, these compounds also comprise as monomer an ester of an α,β-monoethylenically unsaturated carboxylic acid and of a C₁-C₄ alcohol.

Examples of compounds of this type that may be mentioned include Aculyn 22® sold by the company Röhm & Haas, which is a methacrylic acid/ethyl acrylate/oxyalkylenated stearyl methacrylate (comprising 20 EU units) terpolymer or Aculyn 28® (methacrylic acid/ethyl acrylate/oxyethylenated behenyl methacrylate (25 EO) terpolymer).

Associative anionic polymers that may also be mentioned include anionic polymers comprising at least one hydrophilic unit of unsaturated olefinic carboxylic acid type, and at least one hydrophobic unit exclusively of the type such as a (C₁₀-C₃₀) alkyl ester of an unsaturated carboxylic acid. Examples that may be mentioned include the anionic polymers described and prepared according to patents U.S. Pat. No. 3,915,921 and U.S. Pat. No. 4,509,949.

Associative anionic polymers that may also be mentioned include anionic terpolymers.

The anionic terpolymer used according to the invention is a linear or branched and/or crosslinked terpolymer, of at least one monomer (1) bearing an acid function in free form, which is partially or totally salified with a nonionic monomer (2) chosen from N,N-dimethylacrylamide and 2-hydroxyethyl acrylate and at least one polyoxyethylenated alkyl acrylate monomer (3) of formula (I) below:

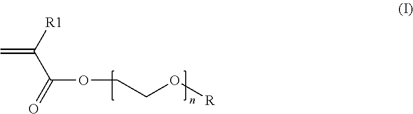

in which R1 represents a hydrogen atom, R represents a linear or branched C₂-C₈ alkyl radical and n represents a number ranging from 1 to 10.

The term “branched polymer” denotes a non-linear polymer which bears pendent chains so as to obtain, when this polymer is dissolved in water, a high degree of entanglement leading to very high viscosities, at a low speed gradient.

The term “crosslinked polymer” denotes a non-linear polymer which is in the form of a three-dimensional network that is insoluble in water but swellable in water, leading to the production of a gel.

The acid function of the monomer (1) is especially a sulfonic acid or phosphonic acid function, said functions being in free or partially or totally salified form.

The monomer (1) may be chosen from styrenesulfonic acid, ethylsulfonic acid and 2-methyl-2-[(1-oxo-2-propenyl]amino]-1-propanesulfonic acid (also known as acryloyldimethyl taurate), in free or partially or totally salified form. It is present in the anionic terpolymer preferably in molar proportions of between 5 mol % and 95 mol % and more particularly between 10 mol % and 90 mol %. The monomer (1) will more particularly be 2-methyl-2-[(1-oxo-2-propenyl)amino]-1-propanesulfonic acid in free or partially or totally salified form.

The acid function in partially or totally salified form will preferably be an alkali metal salt such as a sodium or potassium salt, an ammonium salt, an amino alcohol salt such as a monoethanolamine salt, or an amino acid salt such as a lysine salt.

The monomer (2) is preferably present in the anionic terpolymer in molar proportions of between 4.9 mol % and 90 mol %, more particularly between 9.5 mol % and 85 mol % and even more particularly between 19.5 mol % and 75 mol %.

In formula (I), examples of linear C₈-C₁₆ alkyl radicals that may be mentioned include octyl, decyl, undecyl, tridecyl, tetradecyl, pentadecyl and hexadecyl.

In formula (I), examples of branched C₈-C₁₆ alkyl radicals that may be mentioned include 2-ethylhexyl, 2-propylheptyl, 2-butyloctyl, 2-pentylnonyl, 2-hexyldecyl, 4-methylpentyl, 5-methylhexyl, 6-methylheptyl, 15-methylpentadecyl, 16-methylheptadecyl and 2-hexyloctyl.

According to a particular form of the invention, in formula (I), R denotes a C₁₂-C₁₆ alkyl radical.

According to a particular form of the invention, in formula (I), n ranges from 3 to 5.

Tetraethoxylated lauryl acrylate will more particularly be used as monomer of formula (I).

The monomer (3) of formula (I) is preferably present in the anionic terpolymer in molar proportions of between 0.1 mol % and 10 mol % and more particularly between 0.5 mol % and 5 mol %.

According to a particular mode of the invention, the anionic terpolymer is crosslinked and/or branched with a diethylenic or polyethylenic compound in the proportion expressed relative to the total amount of monomers used, from 0.005 mol % to 1 mol %, preferably from 0.01 mol % to 0.5 mol % and more particularly from 0.01 mol % to 0.25 mol %.

The crosslinking agent and/or branching agent is preferably chosen from ethylene glycol dimethacrylate, diallyloxyacetic acid or a salt thereof, such as sodium diallyloxyacetate, tetraallyloxyethane, ethylene glycol diacrylate, diallylurea, triallylamine, trimethylolpropane triacrylate and methylenebis(acrylamide), or mixtures thereof.

The anionic terpolymer may contain additives such as complexing agents, transfer agents or chain-limiting agents.

Use will be made more particularly of an anionic terpolymer of 2-methyl-2-[(1-oxo-2-propenyl)amino]-1-propanesulfonic acid partially or totally salified in the form of the ammonium salt, N,N-dimethylacrylamide and tetraethoxylated lauryl acrylate crosslinked with trimethylolpropane triacrylate, of INCI name Polyacrylate Crosspolymer-6, such as the product sold under the trade name Sepimax Zen® by the company SEPPIC.

Cationic Associative Polymers

Cationic associative polymers that may be mentioned include polyacrylates bearing amine side groups.

The polyacrylates bearing quaternized or non-quaternized amino side groups contain, for example, hydrophobic groups of the type such as steareth-20 (polyoxyethylenated (20) stearyl alcohol).

Examples of polyacrylates bearing amino side chains that may be mentioned are the polymers 8781-121B or 9492-103 from the company National Starch.

Nonionic Associative Polymers

The nonionic associative polymers may be chosen from:

copolymers of vinylpyrrolidone and of fatty-chain hydrophobic monomers;

copolymers of C₁-C₆ alkyl methacrylates or acrylates and of amphiphilic monomers comprising at least one fatty chain;

copolymers of hydrophilic methacrylates or acrylates and of hydrophobic monomers comprising at least one fatty chain, for instance the polyethylene glycol methacrylate/lauryl methacrylate copolymer; and

associative polyurethanes.

Associative polyurethanes are nonionic block copolymers comprising in the chain both hydrophilic blocks usually of polyoxyethylene nature (polyurethanes may also be referred to as polyurethane polyethers), and hydrophobic blocks that may be aliphatic sequences alone and/or cycloaliphatic and/or aromatic sequences.

In particular, these polymers comprise at least two hydrocarbon-based lipophilic chains containing from 6 to 30 carbon atoms, separated by a hydrophilic block, the hydrocarbon-based chains possibly being pendent chains or chains at the end of the hydrophilic block. In particular, it is possible for one or more pendent chains to be envisaged. In addition, the polymer may comprise a hydrocarbon-based chain at one end or at both ends of a hydrophilic block.

Associative polyurethanes may be block polymers, in triblock or multiblock form. The hydrophobic blocks may thus be at each end of the chain (for example: triblock copolymer containing a hydrophilic central block) or distributed both at the ends and in the chain (for example: multiblock copolymer). These polymers may also be graft polymers or star polymers. Preferably, the associative polyurethanes are triblock copolymers in which the hydrophilic block is a polyoxyethylene chain comprising from 50 to 1000 oxyethylene groups. In general, associative polyurethanes comprise a urethane bond between the hydrophilic blocks, whence arises the name.

According to one preferred embodiment, a nonionic associative polymer of polyurethane type is used as gelling agent.

As examples of nonionic fatty-chain polyurethane polyethers that may be used in the invention, it is also possible to use Rheolate® FX 1100 (Steareth-100/PEG 136/HDI (hexamethyl diisocyanate) copolymer), Rheolate® 205 containing a urea function, sold by the company Elementis, or Rheolate® 208, 204 or 212, and also Acrysol® RM 184 or Acrysol® RM 2020.

Mention may also be made of the product Elfacos® T210 containing a C₁₂-C₁₄ alkyl chain, and the product Elfacos® T212 containing a C₁₆₋₁₈ alkyl chain (PPG-14 Palmeth-60 Hexyl Dicarbamate), from Akzo.

The product DW 1206B® from Röhm & Haas containing a C₂₀ alkyl chain and a urethane bond, sold at a solids content of 20% in water, may also be used.

Use may also be made of solutions or dispersions of these polymers. Examples of such polymers that may be mentioned are Rheolate® 255, Rheolate® 278 and Rheolate® 244 sold by the company Elementis. The products DW 1206F and DW 1206J sold by the company Röhm & Haas may also be used.

The associative polyurethanes that may be used according to the invention are in particular those described in the article by G. Fonnum, J. Bakke and Fk. Hansen, Colloid Polym. Sci., 271, 380-389 (1993).

Even more particularly, according to the invention, use may also be made of an associative polyurethane that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold in particular by the company Röhm & Haas under the names Aculyn® 46 and Aculyn® 44. Aculyn® 46 is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%), and Aculyn® 44 is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%).

Use may also be made of solutions or dispersions of these polymers. Examples of such polymers that may be mentioned include SER AD FX1010, SER AD FX1035 and SER AD 1070 from the company Elementis. Use may also be made of the products Aculyn® 44, Aculyn® 46, DW 1206F and DW 1206J, and also Acrysol® RM 184 from the company Röhm & Haas, or alternatively Borchigel LW 44 from the company Borchers, and mixtures thereof.

The non-ionic associative polymers are advantageously used in a proportion of from 0.5% to 15% by weight of solids and preferably between 1% and 10% by weight, relative to the total weight of the composition.

Amphoteric Associative Polymers

Among the associative amphoteric polymers of the invention, mention may be made of crosslinked or non-crosslinked, branched or unbranched amphoteric polymers, which may be obtained by copolymerization:

1) of at least one monomer of formula (IVa) or (IVb):

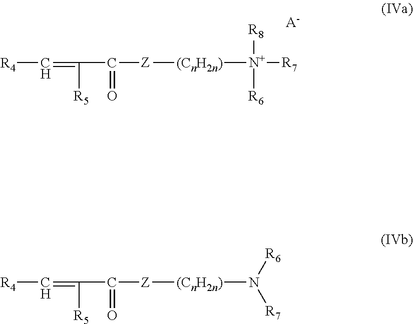

in which R₄ and R₅, which may be identical or different, represent a hydrogen atom or a methyl radical,

R₆, R₇ and R₈, which may be identical or different, represent a linear or branched alkyl radical containing from 1 to 30 carbon atoms;

Z represents an NH group or an oxygen atom;

n is an integer from 2 to 5;

A⁻ is an anion derived from a mineral or organic acid, such as a methosulfate anion or a halide such as chloride or bromide;

2) of at least one monomer of formula (V):

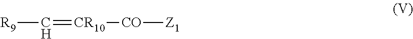

in which R₉ and R₁₀, which may be identical or different, represent a hydrogen atom or a methyl radical;

Z₁ represents a group OH or a group NHC(CH₃)₂CH₂SO₃H;

3) of at least one monomer of formula (VI):

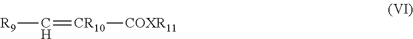

in which R₉ and R₁₀, which may be identical or different, represent a hydrogen atom or a methyl radical, X denotes an oxygen or nitrogen atom and R₁₁ denotes a linear or branched alkyl radical containing from 1 to 30 carbon atoms;

4) optionally at least one crosslinking or branching agent; at least one of the monomers of formula (IVa), (IVb) or (VI) comprising at least one fatty chain containing from 8 to 30 carbon atoms and said compounds of the monomers of formulae (IVa), (IVb), (V) and (VI) possibly being quaternized, for example with a C₁-C₄ alkyl halide or a C₁-C₄ dialkyl sulfate.

The monomers of formulae (IVa) and (IVb) of the present invention are preferably chosen from the group consisting of:

dimethylaminoethyl methacrylate, dimethylaminoethyl acrylate,

diethylaminoethyl methacrylate, diethylaminoethyl acrylate,

dimethylaminopropyl methacrylate, dimethylaminopropyl acrylate,

dimethylaminopropylmethacrylamide, dimethylaminopropylacrylamide,

which are optionally quaternized, for example with a C₁-C₄ alkyl halide or a C₁-C₄ dialkyl sulfate.

More particularly, the monomer of formula (IVa) is chosen from acrylamidopropyltrimethylammonium chloride and methacrylamidopropyl-trimethylammonium chloride.

The compounds of formula (V) of the present invention are preferably chosen from the group formed by acrylic acid, methacrylic acid, crotonic acid, 2-methylcrotonic acid, 2-acrylamido-2-methylpropanesulfonic acid and 2-methacrylamido-2-methylpropanesulfonic acid. More particularly, the monomer of formula (V) is acrylic acid.

The monomers of formula (VI) of the present invention are preferably chosen from the group formed by C₁₂-C₂₂ and more particularly C₁₆-C₁₈ alkyl acrylates or methacrylates.

The crosslinking or branching agent is preferably chosen from N,N′-methylenebisacrylamide, triallylmethylammonium chloride, allyl methacrylate, n-methylolacrylamide, polyethylene glycol dimethacrylates, ethylene glycol dimethacrylate, diethylene glycol dimethacrylate, 1,6-hexanediol dimethacrylate and allyl sucrose.

The polymers according to the invention may also contain other monomers such as nonionic monomers and in particular such as C₁-C₄ alkyl acrylates or methacrylates.

The ratio of the number of cationic charges/anionic charges in these amphoteric polymers is preferably equal to about 1

The weight-average molecular weights of the associative amphoteric polymers have a weight-average molecular mass of greater than 500 g/mol, preferably between 10 000 g/mol and 10 000 000 g/mol and even more preferentially between 100 000 g/mol and 8 000 000 g/mol.

Preferably, the associative amphoteric polymers of the invention contain from 1 mol % to 99 mol %, more preferentially from 20 mol % to 95 mol % and even more preferentially from 25 mol % to 75 mol % of compound(s) of formula (IVa) or (IVb). They also preferably contain from 1 mol % to 80 mol %, more preferentially from 5 mol % to 80 mol % and even more preferentially from 25 mol % to 75 mol % of compound(s) of formula (V). The content of compound(s) of formula (VI) is preferably between 0.1 mol % and 70 mol %, more preferentially between 1 mol % and 50 mol % and even more preferentially between 1 mol % and 10 mol %. The crosslinking or branching agent, when it is present, is preferably between 0.0001 mol % and 1 mol % and even more preferentially between 0.0001 mol % and 0.1 mol %.

Preferably, the mole ratio between the compound(s) of formula (IVa) or (IVb) and the compound(s) of formula (V) ranges from 20/80 to 95/5 and more preferentially from 25/75 to 75/25.

The associative amphoteric polymers according to the invention are described, for example, in patent application WO 98/44012.

The amphoteric polymers that are particularly preferred according to the invention are chosen from acrylic acid/acrylamidopropyltrimethylammonium chloride/stearyl methacrylate copolymers.

Such an associative polymer is advantageously used in a proportion of from 0.1% to 10% by weight of solids and preferably between 0.2% and 6% by weight, relative to the total weight of the composition.

2 Polyacrylamides and 2-acrylamido-2-methylpropanesulfonic Acid Polymers and Copolymers

The polymers used that are suitable as aqueous gelling agent for the invention may be crosslinked or non-crosslinked homopolymers or copolymers comprising at least the 2-acrylamido-2-methylpropanesulfonic acid (AMPS®) monomer, in a form partially or totally neutralized with a mineral base other than aqueous ammonia, such as sodium hydroxide or potassium hydroxide.

They are preferably totally or almost totally neutralized, i.e. at least 90% neutralized.

These AMPS® polymers according to the invention may be crosslinked or non-crosslinked.

When the polymers are crosslinked, the crosslinking agents may be chosen from the polyolefinically unsaturated compounds commonly used for crosslinking polymers obtained by radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allylic or vinyl ethers of polyfunctional alcohols, and also the allylic esters of phosphoric and/or vinylphosphonic acid derivatives, or mixtures of these compounds.

According to one preferred embodiment of the invention, the crosslinking agent is chosen from methylenebisacrylamide, allyl methacrylate and trimethylolpropane triacrylate (TMPTA). The degree of crosslinking generally ranges from 0.01 mol % to 10 mol % and more particularly from 0.2 mol % to 2 mol % relative to the polymer.

The AMPS® polymers that are suitable for use in the invention are water-soluble or water-dispersible. In this case, they are:

either “homopolymers” comprising only AMPS monomers and, if they are crosslinked, one or more crosslinking agents such as those defined above;

or copolymers obtained from AMPS® and from one or more hydrophilic or hydrophobic ethylenically unsaturated monomers and, if they are crosslinked, one or more crosslinking agents such as those defined above. When said copolymers comprise hydrophobic ethylenically unsaturated monomers, these monomers do not comprise a fatty chain and are preferably present in small amounts.

For the purpose of the present invention, the term “fatty chain” is intended to mean any hydrocarbon-based chain comprising at least 7 carbon atoms.

The term “water-soluble or water-dispersible” means polymers which, when introduced into an aqueous phase at 25° C., at a mass concentration equal to 1%, make it possible to obtain a macroscopically homogeneous and transparent solution, i.e. a solution with a maximum light transmittance value, at a wavelength equal to 500 nm, through a sample 1 cm thick, of at least 60% and preferably of at least 70%.

The “homopolymers” according to the invention are preferably crosslinked and neutralized, and they may be obtained according to the preparation process comprising the following steps:

(a) the monomer such as AMPS in free form is dispersed or dissolved in a solution of tert-butanol or of water and tert-butanol;

(b) the monomer solution or dispersion obtained in (a) is neutralized with one or more mineral or organic bases, preferably aqueous ammonia NH₃, in an amount making it possible to obtain a degree of neutralization of the sulfonic acid functions of the polymer ranging from 90% to 100%;

(c) the crosslinking monomer(s) are added to the solution or dispersion obtained in (b);

(d) a standard free-radical polymerization is performed in the presence of free-radical initiators at a temperature ranging from 10° C. to 150° C.; the polymer precipitates from the tert-butanol-based solution or dispersion.

The water-soluble or water-dispersible AMPS® copolymers according to the invention contain water-soluble ethylenically unsaturated monomers, hydrophobic monomers, or mixtures thereof.

The water-soluble comonomers may be ionic or nonionic.

Among the ionic water-soluble comonomers, examples that may be mentioned include the following compounds, and salts thereof:

(meth)acrylic acid,

styrenesulfonic acid,

vinylsulfonic acid and (meth)allylsulfonic acid,

vinylphosphonic acid,

maleic acid,

itaconic acid,

crotonic acid,

water-soluble vinyl monomers of formula (A) below:

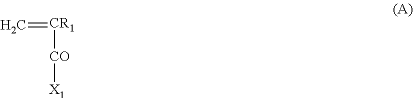

in which:

R₁ is chosen from H, —CH₃, —C₂H₅ and —C₃H₇,

X₁ is chosen from:

alkyl oxides of type —OR₂ where R₂ is a linear or branched, saturated or unsaturated hydrocarbon-based radical containing from 1 to 6 carbon atoms, substituted with at least one sulfonic (—SO₃—) and/or sulfate (—SO₄—) and/or phosphate (—PO₄H₂—) group.

Among the nonionic water-soluble comonomers, examples that may be mentioned include:

(meth)acrylamide,

N-vinylacetamide and N-methyl-N-vinylacetamide,

N-vinylformamide and N-methyl-N-vinylformamide,

maleic anhydride,

vinylamine,

N-vinyllactams comprising a cyclic alkyl group containing from 4 to 9 carbon atoms, such as N-vinylpyrrolidone, N-butyrolactam and N-vinylcaprolactam,

vinyl alcohol of formula CH₂═CHOH,

water-soluble vinyl monomers of formula (B) below:

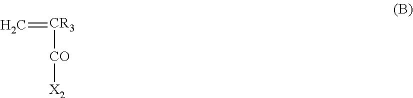

in which:

R₃ is chosen from H, —CH₃, —C₂H₅ and —C₃H₇,

X₂ is chosen from alkyl oxides of the type —OR₄ where R₄ is a linear or branched, saturated or unsaturated hydrocarbon-based radical containing from 1 to 6 carbon atoms, optionally substituted with a halogen (iodine, bromine, chlorine or fluorine) atom; a hydroxyl (—OH) group; ether.

Mention is made, for example, of glycidyl (meth)acrylate, hydroxyethyl methacrylate, and (meth)acrylates of ethylene glycol, of diethylene glycol or of polyalkylene glycol.

Among the hydrophobic co-monomers without a fatty chain, mention may be made, for example, of:

styrene and derivatives thereof, such as 4-butylstyrene, α-methylstyrene and vinyltoluene;

vinyl acetate of formula CH₂═CH—OCOCH₃;

vinyl ethers of formula CH₂═CHOR in which R is a linear or branched, saturated or unsaturated hydrocarbon-based radical containing from 1 to 6 carbons;

acrylonitrile;

caprolactone;

vinyl chloride and vinylidene chloride;

silicone derivatives, which, after polymerization, result in silicone polymers such as methacryloxypropyltris(trimethylsiloxy)silane and silicone methacrylamides;

hydrophobic vinyl monomers of formula (C) below:

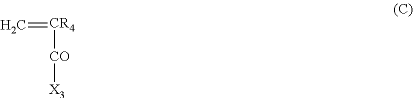

in which:

R₄ is chosen from H, —CH₃, —C₂H₅ and —C₃H₇;

X₃ is chosen from:

alkyl oxides of the type —OR₅ where R₅ is a linear or branched, saturated or unsaturated hydrocarbon-based radical containing from 1 to 6 carbon atoms.

Mention is made, for example, of methyl methacrylate, ethyl methacrylate, n-butyl (meth)acrylate, tert-butyl (meth)acrylate, cyclohexyl acrylate, isobornyl acrylate and 2-ethylhexyl acrylate.

The water-soluble or water-dispersible AMPS® polymers of the invention preferably have a molar mass ranging from 50 000 g/mol to 10 000 000 g/mol, preferably from 80 000 g/mol to 8 000 000 g/mol, and even more preferably from 100 000 g/mol to 7 000 000 g/mol.

As water-soluble or water-dispersible AMPS homopolymers suitable for use in the invention, mention may be made, for example, of crosslinked or non-crosslinked polymers of sodium acrylamido-2-methylpropanesulfonate, such as that used in the commercial product Simulgel 800 (CTFA name: Sodium Polyacryloyldimethyl Taurate), crosslinked ammonium acrylamido-2-methylpropanesulfonate polymers (INCI name: Ammonium Polyacryldimethyltauramide) such as those described in patent EP 0 815 928 B1 and such as the product sold under the trade name Hostacerin AMPS® by the company Clariant.

As preferred water-soluble or water-dispersible AMPS homopolymers in accordance with the invention, mention may be made of ammonium 2-acrylamido-2-methylpropanesulfonate polymers.

As water-soluble or water-dispersible AMPS copolymers in accordance with the invention, examples that may be mentioned include:

crosslinked acrylamide/sodium acrylamido-2-methylpropanesulfonate copolymers, such as that used in the commercial product Sepigel 305® (CTFA name: Polyacrylamide/C₁₃-C₁₄ Isoparaffin/Laureth-7) or that used in the commercial product sold under the name Simulgel 600 (CTFA name: Acrylamide/Sodium acryloyldimethyltaurate/Isohexadecane/Polysorbate-80) by the company SEPPIC;

copolymers of AMPS® and of vinylpyrrolidone or vinylformamide, such as that used in the commercial product sold under the name Aristoflex AVC® by the company Clariant (CTFA name: Ammonium Acryloyldimethyltaurate/VP copolymer) but neutralized with sodium hydroxide or potassium hydroxide;

copolymers of AMPS® and of sodium acrylate, for instance the AMPS/sodium acrylate copolymer, such as that used in the commercial product sold under the name Simulgel EG® by the company SEPPIC;

copolymers of AMPS® and of hydroxyethyl acrylate, for instance the AMPS®/hydroxyethyl acrylate copolymer, such as that used in the commercial product sold under the name Simulgel NS® by the company SEPPIC (CTFA name: Hydroxyethyl acrylate/Sodium acryloyldimethyltaurate copolymer (and) Squalane (and) Polysorbate 60), or such as the product sold under the name Sodium acrylamido-2-methylpropanesulfonate/Hydroxyethyl acrylate copolymer, such as the commercial product Sepinov EMT 10 or under the trade name Sepinov EM (INCI name: Hydroxyethyl acrylate/Sodium acryloyldimethyltaurate copolymer).

As preferred water-soluble or water-dispersible AMPS copolymers in accordance with the invention, mention may be made of copolymers of AMPS® and of hydroxyethyl acrylate.

In general, a composition according to the invention may comprise from 0.1% to 10% by weight, preferably from 0.2% to 8% by weight and more preferentially from 0.2% to 6% by weight of solids of polyacrylamide(s) and/or of crosslinked and/or neutralized 2-acrylamido-2-methylpropanesulfonic acid polymer(s) and copolymer(s) relative to the total weight of the composition.

3 Modified or Unmodified Carboxyvinyl Polymers

The modified or unmodified carboxyvinyl polymers may be homopolymers or copolymers derived from the polymerization of at least one monomer chosen from α,β-ethylenically unsaturated carboxylic acids or esters thereof.

The term “copolymers” means both copolymers obtained from two types of monomer and those obtained from more than two types of monomer, such as terpolymers obtained from three types of monomer.

Their chemical structure more particularly comprises at least one hydrophilic unit and at least one hydrophobic unit. The term “hydrophobic group or unit” means a radical with a saturated or unsaturated, linear or branched hydrocarbon-based chain, comprising at least 8 carbon atoms, preferably from 10 to 30 carbon atoms, in particular from 12 to 30 carbon atoms and more preferentially from 18 to 30 carbon atoms.

Preferably, these copolymers are chosen from copolymers derived from the polymerization:

of at least one monomer of formula (1) below:

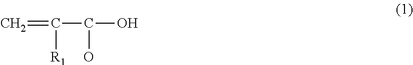

in which R₁ denotes H or CH₃ or C₂H₅, i.e. acrylic acid, methacrylic acid or ethacrylic acid monomers, and

of at least one monomer of unsaturated carboxylic acid (C₁₀-C₃₀)alkyl ester type corresponding to the monomer of formula (2) below:

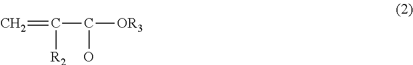

in which R₂ denotes H or CH₃ or C₂H₅ (i.e. acrylate, methacrylate or ethacrylate units) and preferably H (acrylate units) or CH₃ (methacrylate units), R₃ denoting a C₁₀-C₃₀ and preferably C₁₂-C₂₂ alkyl radical.

The unsaturated carboxylic acid (C₁₀-C₃₀)alkyl esters are preferably chosen from lauryl acrylate, stearyl acrylate, decyl acrylate, isodecyl acrylate and dodecyl acrylate, and the corresponding methacrylates, such as lauryl methacrylate, stearyl methacrylate, decyl methacrylate, isodecyl methacrylate and dodecyl methacrylate, and mixtures thereof.

According to a preferred embodiment, these polymers are crosslinked.

Among the copolymers of this type that will be used more particularly are polymers derived from the polymerization of a monomer mixture comprising:

essentially acrylic acid,

an ester of formula (2) described above in which R₂ denotes H or CH₃, R₃ denoting an alkyl radical containing from 12 to 22 carbon atoms, and

a crosslinking agent, which is a well-known copolymerizable unsaturated polyethylenic monomer, such as diallyl phthalate, allyl (meth)acrylate, divinylbenzene, (poly)ethylene glycol dimethacrylate and methylenebisacrylamide.

Among the copolymers of this type, use will more particularly be made of those consisting of from 95% to 60% by weight of acrylic acid (hydrophilic unit), 4% to 40% by weight of C₁₀-C₃₀ alkyl acrylate (hydrophobic unit) and 0 to 6% by weight of crosslinking polymerizable monomer, or alternatively those consisting of from 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described previously.

Among the abovementioned polymers, the ones that are most particularly preferred according to the present invention are acrylate/C₁₀-C₃₀-alkyl acrylate copolymers (INCI name: Acrylates/C₁₀-₃₀ Alkyl acrylate Crosspolymer) such as the products sold by the company Lubrizol under the trade names Pemulen TR-1, Pemulen TR-2, Carbopol 1382, Carbopol EDT 2020 and Carbopol Ultrez 20 Polymer, and even more preferentially Pemulen TR-2.

Among the modified or unmodified carboxyvinyl polymers, mention may also be made of sodium polyacrylates such as those sold under the name Cosmedia SP® containing 90% solids and 10% water, or Cosmedia SPL® as an inverse emulsion containing about 60% solids, an oil (hydrogenated polydecene) and a surfactant (PPG-5 Laureth-5), both sold by the company Cognis.

Mention may also be made of partially neutralized sodium polyacrylates that are in the form of an inverse emulsion comprising at least one polar oil, for example the product sold under the name Luvigel® EM sold by the company BASF. The modified or unmodified carboxyvinyl polymers may also be chosen from crosslinked (meth)acrylic acid homopolymers.

For the purposes of the present patent application, the term “(meth)acrylic” means “acrylic or methacrylic”.

Examples that may be mentioned include the products sold by Lubrizol under the names Carbopol 910, 934, 940, 941, 934 P, 980, 981, 2984, 5984 and Carbopol Ultrez 10 Polymer, or by 3V-Sigma under the name Synthalen® K, Synthalen® L or Synthalen® M.

Among the modified or unmodified carboxyvinyl polymers, mention may be made in particular of Carbopol (INCI name: carbomer) and Pemulen (CTFA name: Acrylates/C₁₀₋₃₀ alkyl acrylate crosspolymer) sold by the company Lubrizol.

The modified or unmodified carboxyvinyl polymers may be present in a proportion of from 0.1% to 5% by weight of solids relative to the weight of the composition, in particular from 0.2% to 4% by weight and preferably between 0.3% and 3% by weight, relative to the weight of the composition.

Advantageously, a composition according to the invention comprises, as hydrophilic gelling agent, at least one gelling agent chosen from associative polymers which are preferably nonionic; 2-acrylamido-2-methylpropanesulfonic acid polymers and copolymers; and mixtures thereof, in particular associative polymers which are preferably nonionic.

According to a preferred variant, the hydrophilic gelling agent is chosen from copolymers of 2-acrylamido-2-methylpropanesulfonic acid and of hydroxyethyl acrylate; ammonium 2-acrylamido-2-methylpropanesulfonate polymers; nonionic associative polyurethanes, in particular fatty-chain nonionic polyurethane polyethers; and mixtures thereof.

II Mixed Silicates

For the purposes of the present invention, the term “mixed silicate” means all silicates of natural or synthetic origin containing several (two or more) types of cations chosen from alkali metals (for example Na, Li, K) or alkaline-earth metals (for example Be, Mg, Ca), transition metals and aluminium.

According to a particular embodiment, the mixed silicate(s) are in the form of solid particles containing at least 10% by weight of at least one silicate relative to the total weight of the particles. In the rest of the present description, these particles are referred to as “silicate particles”.

Preferably, the silicate particles contain less than 1% by weight of aluminium relative to the total weight of the particles. Even more preferably, they contain from 0 to 1% by weight of aluminium relative to the total weight of the particles.

Preferably, the silicate particles contain at least 50% by weight of silicate and better still at least 70% by weight relative to the total weight of the particles. Particles containing at least 90% by weight of silicates, relative to the total weight of the particles, are particularly preferred.

In particular, it is an alkali metal or alkaline-earth metal, aluminium or iron silicate or mixture of silicates.

Preferably, it is sodium, magnesium and/or lithium silicate.

To ensure good cosmetic properties, these silicates are generally in a finely divided form, and in particular in the form of particles with a mean size ranging from 2 nm to 1 μm (from 2 nm to 1000 nm), preferably from 5 nm to 600 nm and even more preferentially from 20 to 250 nm.

The silicate particles may have any form, for example the form of spheres, flakes, needles, platelets, discs, leaflets, or totally random forms. Preferably, the silicate particles are in the form of discs or leaflets.

Thus, the term “mean size” of the particles means the numerical mean size of the largest dimension (length) that it is possible to measure between two diametrically opposite points on an individual particle. The size may be determined, for example, by transmission electron microscopy or by measuring the specific surface area via the BET method or by laser particle size analysis.

When the particles are in the form of discs or leaflets, they generally have a thickness ranging from about 0.5 nm to 5 nm.

The silicate particles may consist of an alloy with metal or metalloid oxides, obtained, for example, by thermal melting of the various constituents thereof. When the particles also comprise such a metal or metalloid oxide, this oxide is preferably chosen from silicon, boron or aluminium oxide.

According to a particular embodiment of the invention, the silicates are phyllosilicates, namely silicates having a structure in which the SiO₄ tetrahedra are organized in leaflets between which the metal cations are enclosed.

The mixed silicates that are suitable for use in the invention may be chosen, for example, from montmorillonites, hectorites, bentonites, beidellite and saponites. According to a preferred embodiment of the invention, the mixed silicates used are more particularly chosen from hectorites and bentonites, and better still from laponites.

A family of silicates that is particularly preferred in the compositions of the present invention is thus the laponite family. Laponites are sodium magnesium silicates also possibly containing lithium, which have a layer structure similar to that of montmorillonites. Laponite is the synthetic form of the natural mineral known as hectorite. The synthetic origin of this family of silicates is of considerable advantage over the natural form, since it allows good control of the composition of the product. In addition, laponites have the advantage of having a particle size that is much smaller than that of the natural minerals hectorite and bentonite.

Laponites that may especially be mentioned include the products sold under the following names: Laponite® XLS, Laponite® XLG, Laponite® RD, Laponite® RDS, Laponite® XL21 (these products are sodium magnesium silicates and sodium lithium magnesium silicates) by the company Rockwood Additives Limited.

Such gelling agents may be used in a proportion of from 0.1% to 10% by weight of solids relative to the total weight of the aqueous phase, especially from 0.1% to 8% by weight and in particular from 0.5% to 5% by weight, relative to the total weight of the aqueous phase.

Lipophilic Gelling Agent

For the purposes of the present invention, the term “lipophilic gelling agent” means a compound that is capable of gelling the oily phase of the compositions according to the invention.

The gelling agent is lipophilic and is thus present in the oily phase of the composition.

The gelling agent is liposoluble or lipodispersible.

As emerges from the foregoing, the gelled oily phase comprises at least one lipophilic gelling agent chosen from polymeric gelling agents, particulate gelling agents, and mixtures thereof.

I. Lipophilic Polymeric Gelling Agents

These gelling agents may be chosen more particularly from hydrocarbon-based block copolymers, polymers containing hydrogen bonding (hydrogen bonding polymers), and mixtures thereof.

I. Hydrocarbon-Based Block Copolymers

The hydrocarbon-based block copolymers of the invention, also known as block copolymers, are preferably soluble or dispersible in the oily phase.

The hydrocarbon-based block copolymer may especially be a diblock, triblock, multiblock, radial or star copolymer, or mixtures thereof.

Such hydrocarbon-based block copolymers are described in patent application US-A-2002/005 562 and in patent U.S. Pat. No. 5,221,534.

The copolymer may contain at least one block whose glass transition temperature is preferably less than 20° C., preferably less than or equal to 0° C., preferably less than or equal to −20° C., more preferably less than or equal to −40° C. The glass transition temperature of said block may be between −150° C. and 20° C., especially between −100° C. and 0° C.

The hydrocarbon-based block copolymer present in the composition according to the invention may be an amorphous copolymer formed by polymerization of an olefin. The olefin may especially be an elastomeric ethylenically unsaturated monomer.

The term “amorphous polymer” means a polymer that does not have a crystalline form.

As examples of olefins, mention may be made of ethylenic carbide monomers, especially bearing one or two ethylenic unsaturations, containing from 2 to 5 carbon atoms, such as ethylene, propylene, butadiene, isoprene or pentadiene.

Advantageously, the hydrocarbon-based block copolymer is an amorphous block copolymer of styrene and of olefin.

Block copolymers comprising at least one styrene block and at least one block comprising units chosen from butadiene, ethylene, propylene, butylene and isoprene or a mixture thereof are especially preferred.

According to a preferred embodiment, the hydrocarbon-based block copolymer is hydrogenated to reduce the residual ethylenic unsaturations after polymerization of the monomers.

In particular, the hydrocarbon-based block copolymer is an optionally hydrogenated copolymer bearing styrene block is and ethylene/C₃-C₄ alkylene blocks.

According to a preferred embodiment, the composition according to the invention comprises at least one diblock copolymer, which is preferably hydrogenated, preferably chosen from styrene-ethylene/propylene copolymers, styrene-ethylene/butadiene copolymers and styrene-ethylene/butylene copolymers. Diblock polymers are sold especially under the name Kraton® G1701E by the company Kraton polymers.

According to another preferred embodiment, the composition according to the invention comprises at least one triblock copolymer, which is preferably hydrogenated, preferably chosen from styrene-ethylene/propylene-styrene copolymers, styrene-ethylene/butadiene-styrene copolymers and styrene-isoprene-styrene copolymers, styrene-butadiene-styrene copolymers. Triblock polymers are especially sold under the names Kraton® G1650, Kraton® D1101, Kraton® D1102 and Kraton® D1160 by the company Kraton Polymers.

According to one embodiment of the present invention, the hydrocarbon-based block copolymer is a styrene-ethylene/butylene-styrene triblock copolymer.

According to a preferred embodiment of the invention, use may be made especially of a mixture of a styrene-butylene/ethylene-styrene triblock copolymer and of a styrene-ethylene/butylene diblock copolymer, especially the products sold under the name Kraton® G1657M by the company Kraton Polymers.

According to another preferred embodiment, the composition according to the invention comprises a styrene-butylene/ethylene-styrene hydrogenated triblock copolymer and an ethylene-propylene-styrene hydrogenated star polymer, such a mixture possibly being especially in isododecane or in another oil. Such mixtures are sold, for example, by the company Penreco under the trade names Versagel® M5960 and Versagel® M5670.

Advantageously, a diblock copolymer such as those described previously is used as hydrocarbon-based block copolymer, in particular a diblock copolymer of styrene-ethylene/propylene or a diblock and triblock mixture, as described previously.

The hydrocarbon-based block copolymer(s) may be present in a content ranging from 0.5% to 15% by weight relative to the total weight of the composition, preferably ranging from 1% to 10% by weight and even more advantageously from 2% to 8% by weight relative to the total weight of the composition.

2. Polymers Containing Hydrogen Bonding (Hydrogen Bonding Polymers)

As representatives of polymers containing hydrogen bonding that are suitable for use in the invention, mention may be made most particularly of polyamides and in particular hydrocarbon-based polyamides and silicone polyamides.

Polyamides

The oily phase of a composition according to the invention may comprise at least one polyamide chosen from hydrocarbon-based polyamides and silicone polyamides, and mixtures thereof, preferably hydrocarbon-based polyamides.

Preferably, the total content of polyamide(s) is between 0.5% and 20% by weight expressed as solids, preferably between 1% and 15% by weight and in particular between 2% and 10% by weight relative to the total weight of the composition.

For the purposes of the invention, the term “polyamide” means a compound containing at least 2 repeating amide units, preferably at least 3 repeating amide units and better still 10 repeating amide units.

a) Hydrocarbon-Based Polyamide

The term “hydrocarbon-based polyamide” means a polyamide formed essentially of, indeed even consisting of, carbon and hydrogen atoms, and optionally of oxygen or nitrogen atoms, and not comprising any silicon or fluorine atoms. It may contain alcohol, ester, ether, carboxylic acid, amine and/or amide groups.

For the purposes of the invention, the term “functionalized chain” means an alkyl chain comprising one or more functional groups or reagents chosen especially from hydroxyl, ether, ester, oxyalkylene and polyoxyalkylene groups.

Advantageously, this polyamide of the composition according to the invention has a weight-average molecular mass of less than 100 000 g/mol especially ranging from 1000 to 100 000 g/mol, in particular less than 50 000 g/mol especially ranging from 1000 to 50 000 g/mol and more particularly ranging from 1000 to 30 000 g/mol, preferably from 2000 to 20 000 g/mol and better still from 2000 to 10 000 g/mol.

This polyamide is insoluble in water, especially at 25° C.

According to a first embodiment of the invention, the polyamide used is a polyamide of formula (I):

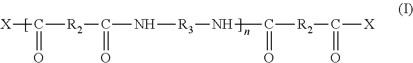

in which X represents a group —N(R₁)₂ or a group —OR₁ in which R₁ is a linear or branched C₈ to C₂₂ alkyl radical which may be identical or different, R₂ is a C₂₈-C₄₂ diacid dimer residue, R₃ is an ethylenediamine radical and n is between 2 and 5; and mixtures thereof.

According to a particular mode, the polyamide used is an amide-terminated polyamide of formula (Ia):

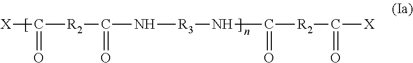

in which X represents a group —N(R₁)₂ in which R₁ is a linear or branched C₈ to C₂₂, alkyl radical which may be identical or different, R₂ is a C₂₈-C₄₂ diacid dimer residue, R₃ is an ethylenediamine radical and n is between 2 and 5;

and mixtures thereof

The oily phase of a composition according to the invention may also comprise, additionally in this case, at least one additional polyamide of formula (Ib):

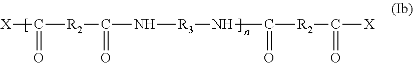

in which X represents a group -OR_{'} in which R₁ is a linear or branched C₈ to C₂₂ and preferably C₁₆ to C₂₂, alkyl radical which may be identical or different, R₂ is a C₂₈-C₄₂ diacid dimer residue, R₃ is an ethylenediamine radical and n is between 2 and 5, such as the commercial products sold by the company Arizona Chemical under the names Uniclear 80 and Uniclear 100 or Uniclear 80 V, Uniclear 100 V and Uniclear 100 VG, the INCI name of which is Ethylenediamine/stearyl dimer dilinoleate copolymer.

b) Silicone Polyamide

The silicone polyamides are preferably solid at room temperature (25° C.) and atmospheric pressure (760 mmHg).

The silicone polyamides may preferentially be polymers comprising at least one unit of formula (III) or (IV)

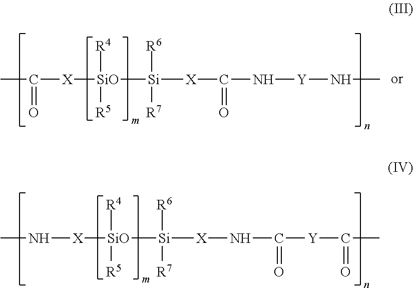

in which:

R⁴, R⁵, R⁶ and R⁷, which may be identical or different, represent a group chosen from:

saturated or unsaturated, C₁ to C₄₀ linear, branched or cyclic hydrocarbon-based groups, which may contain in their chain one or more oxygen, sulfur and/or nitrogen atoms, and which may be partially or totally substituted with fluorine atoms,

C₆ to C₁₀ aryl groups, optionally substituted with one or more C₁ to C₄ alkyl groups,

polyorganosiloxane chains possibly containing one or more oxygen, sulfur and/or nitrogen atoms,

the groups X, which may be identical or different, represent a linear or branched C₁ to C₃₀ alkylenediyl group, possibly containing in its chain one or more oxygen and/or nitrogen atoms,

Y is a saturated or unsaturated C₁ to C₅₀ linear or branched alkylene, arylene, cycloalkylene, alkylarylene or arylalkylene divalent group, which may comprise one or more oxygen, sulfur and/or nitrogen atoms, and/or may bear as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with one to three C₁ to C₃ alkyl, C₁ to C₃ hydroxyalkyl and C₁ to C₆ aminoalkyl groups, or

Y represents a group corresponding to the formula:

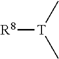

in which

T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and

R⁸ represents a linear or branched C₁ to C₅₀ alkyl group or a polyorganosiloxane chain, possibly comprising one or more ester, amide, urethane, thiocarbamate, urea, thiourea and/or sulfonamide groups, which may possibly be linked to another chain of the polymer,

n is an integer ranging from 2 to 500 and preferably from 2 to 200, and m is an integer ranging from 1 to 1000, preferably from 1 to 700 and even better still from 6 to 200.

According to a particular mode, the silicone polyamide comprises at least one unit of formula (III) in which m ranges from 50 to 200, in particular from 75 to 150 and is preferably about 100.

More preferably, R⁴, R⁵, R⁶ and R⁷ independently represent a linear or branched C₁ to C₄₀ alkyl group, preferably a group CH₃, C₂H₅, n-C₃H₇ or an isopropyl group in formula (III).

As an example of silicone polymers that may be used, mention may be made of one of the silicone polyamides obtained in accordance with Examples 1 to 3 of document U.S. Pat. No. 5,981,680.

Mention may be made of the compounds sold by the company Dow Corning under the names DC 2-8179 (DP 100) and DC 2-8178 (DP 15), the INCI name of which is Nylon-611/dimethicone copolymer, i.e. Nylon-611/dimethicone copolymers. The silicone polymers and/or copolymers advantageously have a temperature of transition from the solid state to the liquid state ranging from 45° C. to 190° C. Preferably, they have a temperature of transition from the solid state to the liquid state ranging from 70° C. to 130° C. and better still from 80° C. to 105° C.

Advantageously, the polymer containing hydrogen bonding is chosen from the ethylenediamine/stearyl dimer dilinoleate copolymer and Nylon-611/dimethicone copolymers.

II. Lipophilic Particulate Gelling Agents

The particulate gelling agent used in the composition according to the invention may be in the form of particles.

As representative lipophilic particulate gelling agents that are suitable for use in the invention, mention may be made most particularly of modified clays and silicas such as fumed silicas and also hydrophobic silica aerogels.

I. Modified Clays

The composition according to the invention may comprise at least one lipophilic clay.

The clays may be natural or synthetic, and they are made lipophilic by treatment with an alkylammonium salt such as a C₁₀ to C₂₂ ammonium chloride, for example distearyldimethylammonium chloride.

They may be chosen from bentonites, in particular hectorites and montmorillonites, beidellites, saponites, nontronites, sepiolites, biotites, attapulgites, vermiculites and zeolites.

They are preferably chosen from hectorites.

Hectorites modified with a C₁₀ to C₂₂ ammonium chloride, such as hectorite modified with distearyldimethylammonium chloride, for instance the product sold under the name Bentone 38V® by the company Elementis or bentone gel in isododecane sold under the name Bentone Gel ISD V® (87% isododecane/10% disteardimonium hectorite/3% propylene carbonate) by the company Elementis, are preferably used as lipophilic clays.

Lipophilic clay may especially be present in a content ranging from 0.1% to 15% by weight, in particular from 0.1% to 10% and more particularly from 0.2% to 8% by weight relative to the total weight of the composition.

2. Silicas

The oily phase of a composition according to the invention may also comprise, as gelling agent, a fumed silica or silica aerogel particles.

a) Fumed Silica

Fumed silica which has undergone a hydrophobic surface treatment is most particularly suitable for use in the invention. Specifically, it is possible to chemically modify the surface of the silica, by chemical reaction generating a reduced number of silanol groups present at the surface of the silica. It is possible in particular to replace silanol groups with hydrophobic groups: a hydrophobic silica is then obtained.

The hydrophobic groups may be:

trimethylsiloxyl groups, which are obtained in particular by treating fumed silica in the presence of hexamethyldisilazane. Silicas thus treated are known as Silica Silylate according to the CTFA (8^{th} edition, 2000). They are sold, for example, under the references Aerosil R812® by the company Degussa and Cab-O-Sil TS-530® by the company Cabot.

dimethylsilyloxyl or polydimethylsiloxane groups, which are obtained in particular by treating fumed silica in the presence of polydimethylsiloxane or dimethyldichlorosilane. Silicas thus treated are known as Silica dimethyl silylate according to the CTFA (8th edition, 2000). They are sold, for example, under the references Aerosil R972® and Aerosil R974® by the company Degussa, and Cab-O-Sil TS-610® and Cab-O-Sil TS-720® by the company Cabot.

The fumed silicas may be present in a composition according to the present invention in a content of between 0.1% and 15% by weight, more particularly between 0.5% and 10% by weight and even more particularly between 1% and 8% by weight relative to the total weight of the composition.

b) Hydrophobic Silica Aerogels

The oily phase of a composition according to the invention may also comprise, as gelling agent, at least silica aerogel particles.

Silica aerogels are porous materials obtained by replacing (by drying) the liquid component of a silica gel with air.

They are generally synthesized via a sol-gel process in a liquid medium and then dried, usually by extraction with a supercritical fluid, the one most commonly used being supercritical CO₂. This type of drying makes it possible to avoid shrinkage of the pores and of the material. The sol-gel process and the various drying operations are described in detail in Brinker C. J. and Scherer G. W., Sol-Gel Science, New York: Academic Press, 1990.

The hydrophobic silica aerogel particles used in the present invention have a specific surface area per unit mass (SM) ranging from 500 to 1500 m²/g, preferably from 600 to 1200 m²/g and better still from 600 to 800 m²/g, and a size expressed as the volume-mean diameter (D[0.5]) ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

According to one embodiment, the hydrophobic silica aerogel particles used in the present invention have a size expressed as volume-mean diameter (D[0.5]) ranging from 1 to 30 μm, preferably from 5 to 25 μm, better still from 5 to 20 μm and even better still from 5 to 15 μm.

The specific surface area per unit mass may be determined by the nitrogen absorption method, known as the BET (Brunauer-Emmett-Teller) method, described in the Journal of the American Chemical Society, vol. 60, page 309, February 1938, which corresponds to International Standard ISO 5794/1 (appendix D). The BET specific surface area corresponds to the total specific surface area of the particles under consideration.

The sizes of the silica aerogel particles may be measured by static light scattering using a commercial particle size analyser such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., Light Scattering by Small Particles, Chapters 9 and 10, Wiley, New York, 1957.

According to an advantageous embodiment, the hydrophobic silica aerogel particles used in the present invention have a specific surface area per unit of mass (SM) ranging from 600 to 800 m²/g.

The silica aerogel particles used in the present invention may advantageously have a tapped density p ranging from 0.02 g/cm³ to 0.10 g/cm³, preferably from 0.03 g/cm³ to 0.08 g/cm³ and in particular ranging from 0.05 g/cm³ to 0.08 g/cm³.

In the context of the present invention, this density, known as the tapped density, may be assessed according to the following protocol:

40 g of powder are poured into a measuring cylinder; the measuring cylinder is then placed on a Stay 2003 machine from Stampf Volumeter; the measuring cylinder is then subjected to a series of 2500 tapping actions (this operation is repeated until the difference in volume between two consecutive tests is less than 2%); the final volume Vf of tapped powder is then measured directly on the measuring cylinder. The tapped density is determined by the ratio m/Vf, in this instance 40/Vf (Vf being expressed in cm³ and m in g).

According to one preferred embodiment, the hydrophobic silica aerogel particles used in the present invention have a specific surface area per unit of volume SV ranging from 5 to 60 m²/cm³, preferably from 10 to 50 m²/cm³ and better still from 15 to 40 m²/cm³.

The specific surface area per unit of volume is given by the relationship: S_{V}=S_{M}×ρ; where ρ is the tapped density, expressed in g/cm³, and S_{M} is the specific surface area per unit of mass, expressed in m²/g, as defined above.

Preferably, the hydrophobic silica aerogel particles according to the invention have an oil-absorbing capacity, measured at the wet point, ranging from 5 to 18 ml/g, preferably from 6 to 15 ml/g and better still from 8 to 12 ml/g.

The absorbing capacity measured at the wet point, noted Wp, corresponds to the amount of oil that needs to be added to 100 g of particles in order to obtain a homogeneous paste.

It is measured according to the wet point method or the method for determining the oil uptake of a powder described in standard NF T 30-022. It corresponds to the amount of oil adsorbed onto the available surface of the powder and/or absorbed by the powder by measurement of the wet point, described below:

An amount m=2 g of powder is placed on a glass plate, and the oil (isononyl isononanoate) is then added dropwise. After addition of 4 to 5 drops of oil to the powder, mixing is carried out using a spatula, and addition of oil is continued until conglomerates of oil and powder have formed. From this point, the oil is added at the rate of one drop at a time and the mixture is subsequently triturated with the spatula. The addition of oil is stopped when a firm, smooth paste is obtained. This paste must be able to be spread on the glass plate without cracking or forming lumps. The volume Vs (expressed in ml) of oil used is then noted.

The oil uptake corresponds to the ratio Vs/m.

The aerogels used according to the present invention are aerogels of hydrophobic silica, preferably of silylated silica (INCI name: silica silylate).

The term “hydrophobic silica” means any silica whose surface is treated with silylating agents, for example with halogenated silanes such as alkylchlorosilanes, siloxanes, in particular dimethylsiloxanes such as hexamethyldisiloxane, or silazanes, so as to functionalize the OH groups with silyl groups Si—Rn, for example trimethylsilyl groups.

As regards the preparation of hydrophobic silica aerogel particles that have been surface-modified by silylation, reference may be made to document U.S. Pat. No. 7,470,725.

Use will preferably be made of hydrophobic silica aerogel particles surface-modified with trimethylsilyl groups, preferably of the INCI name Silica silylate.

As hydrophobic silica aerogels that may be used in the invention, an example that may be mentioned is the aerogel sold under the name VM-2260 or VM-2270 (INCI name: Silica silylate) by the company Dow Corning, the particles of which have a mean size of about 1000 microns and a specific surface area per unit of mass ranging from 600 to 800 m²/g.

Mention may also be made of the aerogels sold by the company Cabot under the references Aerogel TLD 201, Aerogel OGD 201 and Aerogel TLD 203, Enova® Aerogel MT 1100 and Enova Aerogel MT 1200.

Use will preferably be made of the aerogel sold under the name VM-2270 (INCI name: Silica silylate) by the company Dow Corning, the particles of which have an average size ranging from 5-15 microns and a specific surface area per unit of mass ranging from 600 to 800 m²/g.

Preferably, the hydrophobic silica aerogel particles are present in the composition according to the invention in a solids content ranging from 0.1% to 15% by weight, preferably from 0.2% to 10% by weight and preferably from 1% to 8% by weight relative to the total weight of the composition.

According to an advantageous variant, a composition according to the invention comprises, as a lipophilic gelling agent, at least one gelling agent chosen from hydrocarbon-based block copolymers, polymers containing hydrogen bonding such as polyamides, modified clays, and mixtures thereof.

Preferably, the lipophilic gelling agent is chosen from copolymers containing styrene blocks and ethylene/C₃-C₄ alkylene blocks, which are preferably hydrogenated; hydrocarbon-based polyamides; bentonites, in particular hectorites; and mixtures thereof.

Hydrophilic Gelling Agent/Lipophilic Gelling Agent System

The hydrophilic gelling agent(s) according to the invention is (are) advantageously chosen from synthetic polymeric gelling agents.

As preferred synthetic polymeric gelling agents, mention may be made more particularly of:

a) 2-acrylamido-2-methylpropanesulfonic acid polymers, for instance AMPS, such as the ammonium 2-acrylamido-2-methylpropanesulfonate polymer sold under the trade name Hostacerin AMPS® by the company Clariant, and 2-acrylamido-2-methylpropanesulfonic acid copolymers and in particular copolymers of AMPS® and of hydroxyethyl acrylate, for instance the AMPS®/hydroxyethyl acrylate copolymer such as that used in the commercial product sold under the name Simulgel NS® by the company SEPPIC (CTFA name: Hydroxyethyl acrylate/Sodium acryloyldimethyltaurate copolymer (and) Squalane (and) Polysorbate 60), or such as the product sold under the name Sodium acrylamido-2-methylpropanesulfonate/Hydroxyethyl acrylate copolymer, such as the commercial product Sepinov EMT 10 (INCI name: Hydroxyethyl acrylate/Sodium acryloyldimethyltaurate copolymer);

b) associative polymers, in particular nonionic associative polymers, especially of polyurethane type, for instance associative polyurethanes, in particular fatty-chain nonionic polyurethane polyethers such as the Steareth-100/PEG-136/HDI copolymer sold under the name Rheolate FX 1000 by Elementis.

As mentioned previously, a composition according to the invention comprises as lipophilic gelling agent at least one gelling agent chosen from polymeric gelling agents, particulate gelling agents, and mixtures thereof.

Preferred lipophilic gelling agents of polymeric type that may be mentioned include:

polymers containing styrene blocks and ethylene/C₃-C₄ alkylene blocks, which are preferably hydrogenated, such as:
diblock copolymers, which are preferably hydrogenated, chosen especially from styrene-ethylene/propylene copolymers, styrene-ethylene/butadiene copolymers and styrene-ethylene/butylene copolymers, such as the diblock polymers sold under the name Kraton® G1701E by the company Kraton Polymers,
triblock copolymers, which are preferably hydrogenated, preferably chosen from styrene-ethylene/propylene-styrene copolymers, styrene-ethylene/butadiene-styrene copolymers, styrene-isoprene-styrene copolymers and styrene-butadiene-styrene copolymers such as those sold under the names Kraton® G1650, Kraton® D1101, Kraton® D1102 and Kraton® D1160 by the company Kraton Polymers,
mixtures of a styrene-butylene/ethylene-styrene triblock copolymer and of a styrene-ethylene/butylene diblock copolymer, especially those sold under the name Kraton® G1657M by the company Kraton polymers, and
mixtures of styrene-butylene/ethylene-styrene hydrogenated triblock copolymer and of ethylene-propylene-styrene hydrogenated star polymer, such as those sold by the company Penreco under the trade names Versagel® M5960 and Versagel® M5670; and

hydrocarbon-based polyamides such as those sold by the company Arizona chemical under the names Uniclear 80 and Uniclear 100 or Uniclear 80 V, Uniclear 100 V and Uniclear 100 VG, the INCI name of which is ethylenediamine/stearyl dimer dilinoleate copolymer.

As preferred particulate gelling agents, mention may be made of modified clays such as bentonites and preferably hectorites. Bentone 38V® by the company Elementis or the Bentone gel in isododecane sold under the name Bentone Gel ISD V® (87% isododecane/10% disteardimonium hectorite/3% propylene carbonate) by the company Elementis is especially suitable in this respect.

As non-limiting illustrations of the hydrophilic gelling agent/lipophilic gelling agent systems that are most particularly suitable for use in the invention, mention may be made especially of the synthetic polymeric gelling agent/polymeric gelling agent system or the synthetic polymeric gelling agent/particulate gelling agent system.

Thus, a composition according to the invention may advantageously comprise as hydrophilic gelling agent/lipophilic gelling agent system a system chosen from:

nonionic associative polyurethane(s)/hydrocarbon-based block copolymer(s);

nonionic associative polyurethane(s)/polymer(s) containing hydrogen bonding;

nonionic associative polyurethane(s)/modified clays(s);

copolymer(s) of 2-acrylamido-2-methylpropanesulfonic acid and of hydroxyethyl acrylate/hydrocarbon-based block copolymer(s);

copolymer(s) of 2-acrylamido-2-methylpropanesulfonic acid and of hydroxyethyl acrylate/polymer(s) containing hydrogen bonding;

copolymer(s) of 2-acrylamido-2-methylpropanesulfonic acid and of hydroxyethyl acrylate/modified clay(s);

polymer(s) of ammonium 2-acrylamido-2-methylpropanesulfonate/hydrocarbon-based block copolymer(s);

polymer(s) of ammonium 2-acrylamido-2-methylpropanesulfonate/polymer(s) with hydrogen bonding; and

polymer(s) of ammonium 2-acrylamido-2-methylpropanesulfonate/modified clay(s).

Tackifying Resin

As mentioned previously, the claimed compositions comprise at least one tackifying resin, and especially as detailed below.

This type of compound is particularly advantageous insofar as it makes it possible to increase significantly the wear property over time.

In particular, said tackifying resin(s) are present in all or part, and preferably only, in the gelled oily phase.

Preferably, the resin used in the composition according to the invention has a number-average molecular weight of less than or equal to 10 000 g/mol, especially ranging from 250 to 5000 g/mol, better still less than or equal to 2000 g/mol, especially ranging from 250 to 2000 g/mol.

The number-average molecular weights (Mn) are determined by gel permeation liquid chromatography (THF solvent, calibration curve established with linear polystyrene standards, refractometric detector).

Resins that may be suitable for use in the invention are described especially in the Handbook of Pressure Sensitive Adhesive, edited by Donatas Satas, 3rd edition, 1989, pp. 609-619.

The tackifying resin of the composition according to the invention may be chosen from rosin, rosin derivatives and hydrocarbon-based resins, and mixtures thereof, preferably from hydrocarbon-based resins.

Rosin is a mixture predominantly comprising organic acids known as rosin acids (mainly acids of abietic type and of pimaric type).

Three types of rosin exist: rosin (“gum rosin”) obtained by incision on live trees, wood rosin, which is extracted from pine wood or stumps, and tall oil (“tall oil rosin”), which is obtained from a by-product originating from the production of paper.

Rosin derivatives may be derived in particular from the polymerization, hydrogenation and/or esterification of rosin acids, (for example with polyhydric alcohols such as ethylene glycol, glycerol or pentaerythritol). Examples that may be mentioned include the rosin esters sold under the reference Foral 85, Pentalyn H and Staybelite Ester 10 by the company Hercules; Foral 105 Synthetic Resin by the company Pinova; Sylvatac 95 and Zonester 85 by the company Arizona Chemical, or Unirez 3013 by the company Union Camp.

The hydrocarbon-based tackifying resins are preferably chosen from low molecular weight polymers that may be classified, according to the type of monomer they comprise, as:

indene hydrocarbon-based resins, preferably such as the resins derived from the polymerization in major proportion of indene monomer and in minor proportion of a monomer chosen from styrene, methylindene and methylstyrene, and mixtures thereof. These resins may optionally be hydrogenated. These resins may have a molecular weight ranging from 290 to 1150 g/mol;

Examples of indene resins that may be mentioned include those sold under the reference Escorez 7105 by the company Exxon Chem., Nevchem 100 and Nevex 100 by the company Neville Chem., Norsolene S105 by the company Sartomer, Picco 6100 by the company Hercules and Resinall by the company Resinall Corp., or the indene/methylstyrene/hydrogenated styrene copolymers sold under the name Regalite by the company Eastman Chemical, in particular Regalite R1100, Regalite R1090, Regalite R7100, Regalite R1010 Hydrocarbon Resin and Regalite R1125 Hydrocarbon Resin;

aliphatic pentanediene resins, for instance those derived from the majority polymerization of the 1,3-pentanediene (trans or cis-piperylene) monomer and of a minor monomer chosen from isoprene, butene, 2-methyl-2-butene, pentene and 1,4-pentanediene, and mixtures thereof. These resins may have a molecular weight ranging from 1000 to 2500 g/mol;

Such 1,3-pentanediene resins are sold, for example, under the references Piccotac 95 by the company Eastman Chemical, Escorez 1304 by the company Exxon Chemicals, Nevtac 100 by the company Neville Chem. or Wingtack 95 by the company Goodyear;

mixed resins of pentanediene and of indene, which are derived from the polymerization of a mixture of pentanediene and indene monomers such as those described above, for instance the resins sold under the reference Escorez 2101 by the company Exxon Chemicals, Nevpene 9500 by the company Neville Chem., Hercotac 1148 by the company Hercules, Norsolene A 100 by the company Sartomer, and Wingtack 86, Wingtack Extra and Wingtack Plus by the company Goodyear;

diene resins of cyclopentanediene dimers, for instance those derived from the polymerization of a first monomer chosen from indene and styrene, and of a second monomer chosen from cyclopentanediene dimers such as dicyclopentanediene, methyldicyclopentanediene and other pentanediene dimers, and mixtures thereof. These resins generally have a molecular weight ranging from 500 to 800 g/mol, for instance those sold under the reference Betaprene BR 100 by the company Arizona Chemical Co., Neville LX-685-125 and Neville LX-1000 by the company Neville Chem., Piccodiene 2215 by the company Hercules, Petro-Rez 200 by the company Lawter or Resinall 760 by the company Resinall Corp.;

diene resins of isoprene dimers, such as terpenic resins derived from the polymerization of at least one monomer chosen from α-pinene, β-pinene and limonene, and mixtures thereof. These resins may have a molecular weight ranging from 300 to 2000 g/mol. Such resins are sold, for example, under the names Piccolyte A115 and 5125 by the company Hercules, and Zonarez 7100 or Zonatac 105 Lite by the company Arizona Chem.

Mention may also be made of certain modified resins such as hydrogenated resins, for instance those sold under the name Eastotac C6-C20 Polyolefin by the company Eastman Chemical Co, under the reference Escorez 5300 by the company Exxon Chemicals or the resins Nevillac Hard or Nevroz sold by the company Neville Chem., the resins Piccofyn A-100, Piccotex 100 or Piccovar AP25 sold by the company Hercules or the resin SP-553 sold by the company Schenectady Chemical Co.

According to a preferred embodiment, the tackifying resin is chosen from indene hydrocarbon-based resins, aliphatic pentadiene resins, mixed pentanediene and indene resins, diene resins of cyclopentanediene dimers, diene resins of isoprene dimers, or mixtures thereof.

Preferably, the composition comprises at least one compound chosen from the tackifying resins as described previously, especially from hydrocarbon-based resins, in particular from indene hydrocarbon-based resins, aliphatic pentadiene resins, and mixtures thereof. According to a preferred embodiment, the tackifying resin is chosen from indene hydrocarbon-based resins.

According to a preferred embodiment, the resin is chosen from indene/methylstyrene/hydrogenated styrene copolymers.

In particular, products may be made of indene/methylstyrene/hydrogenated styrene copolymers, such as those sold under the name Regalite by the company Eastman Chemical, such as Regalite R 1100 CG Hydrocarbon Resin, Regalite R 1100, Regalite R 1090, Regalite R-7100, Regalite R1010 Hydrocarbon Resin and Regalite R1125 Hydrocarbon Resin.

A composition according to the invention may comprise from 1% to 60% by weight, preferably from 5% to 50% by weight and even more preferentially from 8% to 45% by weight of tackifying resin(s), relative to the total weight of the composition.

Hydrophobic Film-Forming Polymers

The gelled oily phase of the claimed compositions may comprise at least one hydrophobic film-forming polymer especially as detailed below.

This type of polymer is particularly advantageous in so far as it makes it possible to significantly increase the wear property of the deposit over time. As indicated previously, the performance of these polymers is advantageously increased by means of using them in a composition according to the invention.

For the purposes of the present invention, the term “hydrophobic film-forming polymer” is intended to denote a film-forming polymer that has no affinity for water and, in this respect, does not lend itself to a formulation in the form of a solute in an aqueous medium. In particular, the term “hydrophobic polymer” means a polymer having a solubility in water at 25° C. of less than 1% by weight.

The term “film-forming polymer” means a polymer that is capable of forming, by itself or in the presence of an auxiliary film-forming agent, a macroscopically continuous deposit on a support, especially on keratin materials, and preferably a cohesive deposit, and better still a deposit whose cohesion and mechanical properties are such that said deposit may be isolable and manipulable in isolation, for example when said deposit is prepared by pouring onto a non-stick surface, for instance a Teflon-coated or silicone-coated surface.

In particular, the hydrophobic film-forming polymer is a polymer chosen from the group comprising:

film-forming polymers that are soluble in an organic solvent medium, in particular liposoluble polymers; this means that the polymer is soluble or miscible in the organic medium and forms a single homogeneous phase when it is incorporated into the medium; and

film-forming polymers that are dispersible in an organic solvent medium, which means that the polymer forms an insoluble phase in the organic medium, the polymer remaining stable and/or compatible once incorporated into this medium. In particular, such polymers may be in the form of non-aqueous dispersions of polymer particles, preferably dispersions in silicone oils or hydrocarbon-based oils; in one embodiment, the non-aqueous polymer dispersions comprise polymer particles stabilized on their surface with at least one stabilizer; these non-aqueous dispersions are often referred to as NADs.

Hydrophobic film-forming polymers that may especially be mentioned include homopolymers and copolymers of a compound bearing an ethylenic unit, acrylic polymers and copolymers, polyurethanes, polyesters, silicone polymers such as polymers bearing a non-silicone organic backbone grafted with monomers containing a polysiloxane, and polyisoprenes.

A composition according to the invention may comprise from 1% to 30% by weight, preferably from 2% to 25% by weight and even more preferentially from 5% to 20% by weight of hydrophobic film-forming polymer(s) relative to the total weight of the composition.

As hydrophobic film-forming polymers that are most particularly suitable for use in the invention, mention may be made especially of lipodispersible film-forming polymers in the form of non-aqueous dispersions of polymer particles, block ethylenic copolymers, vinyl polymers comprising at least one carbosiloxane dendrimer-based unit, silicone acrylate copolymers and mixtures thereof, preferably lipodispersible film-forming polymers in the form of non-aqueous dispersions of polymer particles (NADs).

I. Lipodispersible Film-Forming Polymers in the Form of Non-Aqueous Dispersions of Polymer Particles, also known as NADs

According to another embodiment variant, a composition according to the invention may comprise, as hydrophobic film-forming polymer, at least one polymer chosen from lipodispersible film-forming polymers in the form of non-aqueous dispersions of polymer particles, also known as NADs.

Non-aqueous dispersions of hydrophobic film-forming polymer that may be used include dispersions of particles of a grafted ethylenic polymer, preferably an acrylic polymer, in a liquid oily phase for example, in the form of surface-stabilized particles dispersed in the liquid fatty phase.

The dispersion of surface-stabilized polymer particles may be manufactured as described in document WO 04/055081.

II. Block Ethylenic Copolymer

According to a first embodiment of the invention, the hydrophobic film-forming polymer is a block ethylenic copolymer, containing at least a first block with a glass transition temperature (T_{g}) of greater than or equal to 40° C. and being totally or partly derived from one or more first monomers, which are such that the homopolymer prepared from these monomers has a glass transition temperature of greater than or equal to 40° C., and at least a second block with a glass transition temperature of less than or equal to 20° C. and being derived totally or partly from one or more second monomers, which are such that the homopolymer prepared from these monomers has a glass transition temperature of less than or equal to 20° C., said first block and said second block being connected together via a statistical intermediate segment comprising at least one of said first constituent monomers of the first block and at least one of said second constituent monomers of the second block, and said block copolymer having a polydispersity index I of greater than 2.

Polymers of this type that are suitable for use in the invention are described in document EP 1 411 069.

As an example of such polymers, mention may be made more particularly of Mexomer PAS® (acrylic acid/isobutyl acrylate/isobornyl acrylate copolymer diluted to 50% in isododecane) sold by the company Chimex.

III. Vinyl Polymer Comprising at Least one Carbosiloxane Dendrimer-Based Unit

According to one particular embodiment, a composition used according to the invention may comprise, as hydrophobic film-forming polymer, at least one vinyl polymer comprising at least one carbosiloxane dendrimer-based unit.

The vinyl polymer used according to the invention especially has a backbone and at least one side chain, which comprises a carbosiloxane dendrimer-based unit having a carbosiloxane dendrimer structure.

Vinyl polymers comprising at least one carbosiloxane dendrimer unit as described in applications WO 03/045 337 and EP 963 751 by the company Dow Corning may be used in particular.

The term “carbosiloxane dendrimer structure” in the context of the present invention represents a molecular structure with branched groups of high molecular masses, said structure having high regularity in the radial direction starting from the bond to the backbone. Such carbosiloxane dendrimer structures are described in the form of a highly branched siloxane-silylalkylene copolymer in the laid-open Japanese patent application Kokai 9-171 154.

A vinyl polymer bearing at least one carbosiloxane dendrimer-based unit has a molecular side chain containing a carbosiloxane dendrimer structure, and may be derived from the polymerization of:

(A) from 0 to 99.9 parts by weight of a vinyl monomer; and

(B) from 100 to 0.1 part by weight of a carbosiloxane dendrimer containing a radical-polymerizable organic group, represented by the general formula:

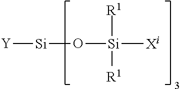

in which Y represents a radical-polymerizable organic group, R¹ represents an aryl group or an alkyl group containing from 1 to 10 carbon atoms, and Xⁱ represents a silylalkyl group which, when i=1, is represented by the formula:

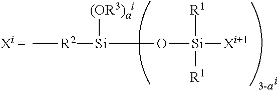

in which R¹ is as defined above, R² represents an alkylene group containing from 2 to 10 carbon atoms, R³ represents an alkyl group containing from 1 to 10 carbon atoms, Xⁱ⁺¹ represents a hydrogen atom, an alkyl group containing from 1 to 10 carbon atoms, an aryl group, or the silylalkyl group defined above with i=i+1; i is an integer from 1 to 10 which represents the generation of said silylalkyl group, and a′ is an integer from 0 to 3;

in which said radical-polymerizable organic group contained in the component (A) is chosen from:

organic groups containing a methacrylic group or an acrylic group and that are represented by the formulae:

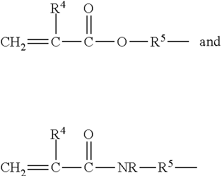

in which R⁴ represents a hydrogen atom or an alkyl group, R⁵ represents an alkylene group containing from 1 to 10 carbon atoms; and

organic groups containing a styryl group and that are represented by the formula:

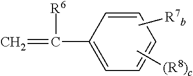

in which R⁶ represents a hydrogen atom or an alkyl group, R⁷ represents an alkyl group containing from 1 to 10 carbon atoms, R⁸ represents an alkylene group containing from 1 to 10 carbon atoms, b is an integer from 0 to 4, and c is 0 or 1, such that if c is 0, —(R⁸)_{c}— represents a bond.

The monomer of vinyl type that is the component (A) in the vinyl polymer is a monomer of vinyl type that contains a radical-polymerizable vinyl group.

There is no particular limitation as regards such a monomer.

The following are examples of this monomer of vinyl type: methyl methacrylate, ethyl methacrylate, n-propyl methacrylate, isopropyl methacrylate or a methacrylate of an analogous lower alkyl; glycidyl methacrylate; butyl methacrylate, butyl acrylate, n-butyl methacrylate, isobutyl methacrylate, tert-butyl acrylate, tert-butyl methacrylate, n-hexyl methacrylate, cyclohexyl methacrylate, 2-ethylhexyl acrylate, 2-ethylhexyl methacrylate, octyl methacrylate, lauryl methacrylate, stearyl acrylate, stearyl methacrylate or a higher-analogue methacrylate; vinyl acetate, vinyl propionate or a vinyl ester of an analogous lower fatty acid; vinyl caproate, vinyl 2-ethylhexoate, vinyl laurate, vinyl stearate or an ester of an analogous higher fatty acid; styrene, vinyltoluene, benzyl methacrylate, phenoxyethyl methacrylate, vinylpyrrolidone or similar vinylaromatic monomers; methacrylamide, N-methylolmethacrylamide, N-methoxymethyl-methacrylamide, isobutoxymethoxymethacrylamide, N,N-dimethylmethacrylamide or similar monomers of vinyl type containing amide groups; hydroxyethyl methacrylate, hydroxypropyl alcohol methacrylate or similar monomers of vinyl type containing hydroxyl groups; acrylic acid, methacrylic acid, itaconic acid, crotonic acid, fumaric acid, maleic acid or similar monomers of vinyl type containing a carboxylic acid group; tetrahydrofurfuryl methacrylate, butoxyethyl methacrylate, ethoxydiethylene glycol methacrylate, polyethylene glycol methacrylate, polypropylene glycol monomethacrylate, hydroxybutyl vinyl ether, cetyl vinyl ether, 2-ethylhexyl vinyl ether or a similar monomer of vinyl type with ether bonds; methacryloxypropyltrimethoxysilane, polydimethylsiloxane containing a methacrylic group on one of its molecular ends, polydimethylsiloxane containing a styryl group on one of its molecular ends, or a similar silicone compound containing unsaturated groups; butadiene; vinyl chloride; vinylidene chloride; methacrylonitrile; dibutyl fumarate; anhydrous maleic acid; anhydrous succinic acid; methacryl glycidyl ether; an organic salt of an amine, an ammonium salt, and an alkali metal salt of methacrylic acid, of itaconic acid, of crotonic acid, of maleic acid or of fumaric acid; a radical-polymerizable unsaturated monomer containing a sulfonic acid group such as a styrenesulfonic acid group; a quaternary ammonium salt derived from methacrylic acid, such as 2-hydroxy-3-methacryloxypropyltrimethylammonium chloride; and a methacrylic acid ester of an alcohol containing a tertiary amine group, such as a methacrylic acid ester of diethylamine.

Multifunctional monomers of vinyl type may also be used.

The following are examples of such compounds: trimethylolpropane trimethacrylate, pentaerythrityl trimethacrylate, ethylene glycol dimethacrylate, tetraethylene glycol dimethacrylate, polyethylene glycol dimethacrylate, 1,4-butanediol dimethacrylate, 1,6-hexanediol dimethacrylate, neopentyl glycol dimethacrylate, trimethylolpropane trioxyethylmethacrylate, tris(2-hydroxyethyl) isocyanurate dimethacrylate, tris(2-hydroxyethyl) isocyanurate trimethacrylate, polydimethylsiloxane capped with styryl groups bearing divinylbenzene groups on the two ends, or similar silicone compounds bearing unsaturated groups.

To facilitate the preparation of starting material mixture for cosmetic products, the number-average molecular mass of the vinyl polymer bearing a carbosiloxane dendrimer may be chosen within the range between 3000 g/mol and 2 000 000 g/mol and preferably between 5000 g/mol and 800 000 g/mol. It may be a liquid, a gum, a paste, a solid, a powder, or any other form. The preferred forms are solutions consisting of the dilution of a dispersion or of a powder in solvents such as a silicone oil or an organic oil.

A vinyl polymer contained in the dispersion or the solution may have a concentration in the range between 0.1% and 95% by weight and preferably between 5% and 70% by weight. However, to facilitate the handling and the preparation of the mixture, the range should preferably be between 10% and 60% by weight.

According to one preferred mode, a vinyl polymer that is suitable for use in the invention may be one of the polymers described in the examples of patent application EP 0 963 751.

According to one preferred embodiment, a vinyl polymer grafted with a carbosiloxane dendrimer may be the product of polymerization of:

(A) from 0.1 to 99 parts by weight of one or more acrylate or methacrylate monomers; and

(B) from 100 to 0.1 part by weight of an acrylate or methacrylate monomer of a tris[tri(trimethylsiloxy)silylethyldimethylsiloxy]silylpropyl carbosiloxane dendrimer.

According to one embodiment, a vinyl polymer bearing at least one carbosilaxane dendrimer-based unit may comprise a tris[tri(trimethylsiloxy)silylethyl-dimethylsiloxy]silylpropyl carbosiloxane dendrimer-based unit corresponding to one of the formulae

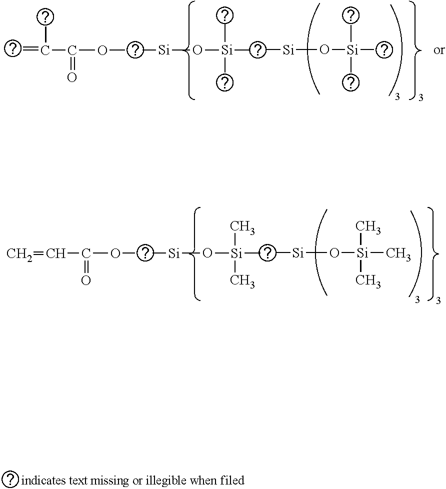

According to one preferred mode, a vinyl polymer bearing at least one carbosiloxane dendrimer-based unit used in the invention comprises at least one butyl acrylate monomer.

According to one embodiment, a vinyl polymer may also comprise at least one fluoro organic group. A fluorinated vinyl polymer may be one of the polymers described in the examples of patent application WO 03/045 337.

According to one preferred embodiment, a vinyl polymer grafted in the sense of the present invention may be conveyed in an oil or a mixture of oils, which is/are preferably volatile, chosen in particular from silicone oils and hydrocarbon-based oils, and mixtures thereof.

According to one particular embodiment, a silicone oil that is suitable for use in the invention may be cyclopentasiloxane.

According to another particular embodiment, a hydrocarbon-based oil that is suitable for use in the invention may be isododecane.

Vinyl polymers grafted with at least one carbosiloxane dendrimer-based unit that may be particularly suitable for use in the present invention are the polymers sold under the names TIB 4-100, TIB 4-101, TIB 4-120, TIB 4-130, TIB 4-200, FA 4002 ID (TIB 4-202), TIB 4-220 and FA 4001 CM (TIB 4-230) by the company Dow Corning. The polymers sold under the names FA 4002 ID (TIB 4-202) and FA 4001 CM (TIB 4-230) by the company Dow Corning will preferably be used.

Preferably, the vinyl polymer grafted with at least one carbosiloxane dendrimer-based unit that may be used in a composition of the invention is an acrylate/polytrimethyl siloxymethacrylate copolymer, especially the product sold in isododecane under the name Dow Corning FA 4002 ID Silicone Acrylate by the company Dow Corning.

IV. Silicone Acrylate Copolymers

According to one particular embodiment, a composition used according to the invention may comprise, as hydrophobic film-forming polymer, at least one copolymer comprising carboxylate groups and polydimethylsiloxane groups.

In the present application, the term “copolymer comprising carboxylate groups and polydimethylsiloxane groups” means a copolymer obtained from (a) one or more carboxylic (acid or ester) monomers, and (b) one or more polydimethylsiloxane (PDMS) chains.

In the present application, the term “carboxylic monomer” means both carboxylic acid monomers and carboxylic acid ester monomers. Thus, the monomer (a) may be chosen, for example, from acrylic acid, methacrylic acid, maleic acid, fumaric acid, itaconic acid, crotonic acid, esters thereof and mixtures of these monomers. Esters that may be mentioned include the following monomers: acrylate, methacrylate, maleate, fumarate, itaconate and/or crotonate. According to one preferred embodiment of the invention, the monomers in ester form are more particularly chosen from linear or branched, preferably C₁-C₂₄ and better still C₁-C₂₂ alkyl acrylates and methacrylates, the alkyl radical preferably being chosen from methyl, ethyl, stearyl, butyl and 2-ethylhexyl radicals, and mixtures thereof.

Thus, according to one particular embodiment of the invention, the copolymer comprises as carboxylate groups at least one group chosen from acrylic acid and methacrylic acid, and methyl, ethyl, stearyl, butyl or 2-ethylhexyl acrylate or methacrylate, and mixtures thereof.

In the present application, the term “polydimethylsiloxanes” (also known as organopolysiloxanes and abbreviated as PDMS) denotes, in accordance with what is generally accepted, any organosilicon polymer or oligomer of linear structure, of variable molecular weight, obtained by polymerization and/or polycondensation of suitably functionalized silanes, and consisting essentially of a repetition of main units in which the silicon atoms are linked together via oxygen atoms (siloxane bond ≡Si—O—Si≡), comprising trimethyl radicals directly linked via a carbon atom to said silicon atoms. The PDMS chains that may be used to obtain the copolymer used according to the invention comprise at least one polymerizable radical group, preferably located on at least one of the ends of the chain, i.e. the PDMS may contain, for example, a polymerizable radical group on the two ends of the chain or one polymerizable radical group on one end of the chain and one trimethylsilyl end group on the other end of the chain. The polymerizable radical group may especially be an acrylic or methacrylic group, in particular a group CH₂═CR₁—CO—O—R₂, in which R₁ represents a hydrogen or a methyl group and R₂ represents —CH₂—, —(CH₂)ₙ— with n=3, 5, 8 or 10, —CH₂—CH(CH₃)—CH₂—, CH₂—CH₂—O—CH₂—CH₂—, —CH₂—CH₂—O—CH₂—CH₂—CH(CH₃)—CH₂—, —CH₂—CH₂—O—CH₂ CH₂—O—CH₂—CH₂1'CH₂—.

The copolymers used in the composition of the invention are generally obtained according to the usual methods of polymerization and grafting, for example by free-radical polymerization (A) of a PDMS comprising at least one polymerizable radical group (for example on one of the ends of the chain or on both ends) and (B) of at least one carboxylic monomer, as described, for example, in documents U.S. Pat. No. 5,061,481 and U.S. Pat. No. 5,219,560.

The copolymers obtained generally have a molecular weight ranging from about 3000 g/mol to 200 000 g/mol and preferably from about 5000 g/mol to 100 000 g/mol.

The copolymer used in the composition of the invention may be in its native form or in dispersed form in a solvent such as lower alcohols containing from 2 to 8 carbon atoms, for instance isopropyl alcohol, or oils, for instance volatile silicone oils (for example cyclopentasiloxane).

As copolymers that may be used in the composition of the invention, mention may be made, for example, of copolymers of acrylic acid and of stearyl acrylate containing polydimethylsiloxane grafts, copolymers of stearyl methacrylate containing polydimethylsiloxane grafts, copolymers of acrylic acid and of stearyl methacrylate containing polydimethylsiloxane grafts, copolymers of methyl methacrylate, butyl methacrylate, 2-ethylhexyl acrylate and stearyl methacrylate containing polydimethylsiloxane grafts. As copolymers that may be used in the composition of the invention, mention may be made in particular of the copolymers sold by the company Shin-Etsu under the names KP-561 (CTFA name: acrylates/dimethicone), KP-541 in which the copolymer is dispersed at 60% by weight in isopropyl alcohol (CTFA name: acrylates/dimethicone and isopropyl alcohol), and KP-545 in which the copolymer is dispersed at 30% in cyclopentasiloxane (CTFA name: acrylates/dimethicone and cyclopentasiloxane). According to one preferred embodiment of the invention, KP561 is preferably used; this copolymer is not dispersed in a solvent, but is in waxy form, its melting point being about 30° C.

Mention may also be made of the grafted copolymer of polyacrylic acid and dimethylpolysiloxane dissolved in isododecane, sold by the company Shin-Etsu under the name KP-550.

Aqueous Phase

The aqueous phase of a composition according to the invention comprises water and optionally a water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the composition of the invention may also be volatile.

Among the water-soluble solvents that may be used in the composition in accordance with the invention, mention may be made especially of lower monoalcohols containing from 1 to 5 carbon atoms such as ethanol and isopropanol, glycols containing from 2 to 8 carbon atoms such as ethylene glycol, propylene glycol, 1,3-butylene glycol and dipropylene glycol, C₃ and C₄ ketones and C₂-C₄ aldehydes.

The aqueous phase (water and optionally the water-miscible solvent) may be present in the composition in a content ranging from 10% to 70%, better still from 15% to 55% by weight and preferably from 20% to 50% by weight relative to the total weight of said composition.

In particular, a composition according to the invention advantageously comprises a water content at least equal to 15% by weight, preferably at least equal to 20% by weight and preferentially ranging from 20% to 70% by weight, relative to the total weight of the composition.

According to another embodiment variant, the aqueous phase of a composition according to the invention may comprise at least one C₂-C₃₂ polyol.

For the purposes of the present invention, the term “polyol' should be understood as meaning any organic molecule comprising at least two free hydroxyl groups.

Preferably, a polyol in accordance with the present invention is present in liquid form at room temperature.

Such polyols may be used in a proportion of from 0.1% to 10% by weight, preferably from 0.2% to 8% by weight and even more preferentially from 0.5% to 6% by weight of C₂-C₃₂ polyol, relative to the total weight of the composition.

The polyols advantageously suitable for the formulation of a composition according to the present invention are those exhibiting in particular from 2 to 32 carbon atoms and preferably from 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols such as glycerol oligomers, for instance diglycerol, and polyethylene glycols, and mixtures thereof.

According to a preferred embodiment of the invention, said polyol is chosen from ethylene glycol, pentaerythritol, trimethylolpropane, propylene glycol, glycerol, polyglycerols, polyethylene glycols and mixtures thereof.

Oily Phase

The oily phase of a composition according to the invention comprises at least one volatile oil and may comprise one or more non-volatile oil(s).

The term “oil' means any fatty substance that is in liquid form at room temperature and atmospheric pressure.

For the purposes of the present invention, the term “non-volatile oil” means an oil with a vapour pressure of less than 0.13 Pa.

For the purposes of the invention, the term “volatile oil' means any oil that is capable of evaporating on contact with the skin in less than one hour, at room temperature and atmospheric pressure. The volatile oil is a volatile cosmetic compound, which is liquid at room temperature, especially having a nonzero vapour pressure, at room temperature and atmospheric pressure, in particular having a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

An oily phase that is suitable for preparing the cosmetic compositions according to the invention may comprise hydrocarbon-based oils, silicone oils, fluoro oils or non-fluoro oils, or mixtures thereof.

An oily phase that is suitable for preparing a composition according to the invention may comprise at least one volatile hydrocarbon-based oil.

For the purposes of the present invention, the term “silicone oil” means an oil comprising at least one silicon atom, and in particular at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

The oils of the invention may be of animal, plant, mineral or synthetic origin. According to one embodiment variant, oils of plant origin are preferred.

Volatile Oils

The volatile oils may be hydrocarbon-based oils or silicone oils.

Among the volatile hydrocarbon-based oils containing from 8 to 16 carbon atoms, mention may be made especially of branched C₈-C₁₆ alkanes, such as C₈-C₁₆ isoalkanes (also known as isoparaffins), isododecane, isodecane, isohexadecane and, for example, the oils sold under the trade names Isopar or Permethyl, branched C₈-C₁₆ esters, such as isohexyl neopentanoate, and mixtures thereof. Preferably, the volatile hydrocarbon-based oil is chosen from volatile hydrocarbon-based oils containing from 8 to 16 carbon atoms, and mixtures thereof, in particular from isododecane, isodecane and isohexadecane, and is especially isododecane.

Mention may also be made of volatile linear alkanes comprising from 8 to 16 carbon atoms, in particular from 10 to 15 carbon atoms and more particularly from 11 to 13 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, mixtures of n-undecane (C₁₁) and of n-tridecane (C₁₃) obtained in Examples 1 and 2 of patent application WO 2008/155 059 from the company Cognis, and mixtures thereof.

Volatile silicone oils that may be mentioned include linear volatile silicone oils such as hexamethyldisiloxane, octamethyltrisiloxane, decamethyltetrasiloxane, tetradecamethylhexasiloxane, hexadecamethylheptasiloxane and dodecamethylpentasiloxane.

Volatile cyclic silicone oils that may be mentioned include hexamethylcyclotrisiloxane, octamethylcyclotetrasiloxane, decamethylcyclopentasiloxane and dodecamethylcyclohexasiloxane.

Preferably, a composition according to the invention comprises at least one hydrocarbon-based oil as volatile oil, in particular isododecane.

More particularly, the volatile oil according to the invention is isododecane.

A composition according to the invention may comprise from 10% to 70% by weight, better still from 15% to 55% by weight and preferably from 18% to 50% by weight of volatile oil(s) relative to the total weight of said composition.

Non-Volatile Oils

The non-volatile oils may be chosen especially from non-volatile hydrocarbon-based, fluoro and/or silicone oils.

Non-volatile hydrocarbon-based oils that may especially be mentioned include:

hydrocarbon-based oils of plant origin, synthetic ethers containing from 10 to 40 carbon atoms, such as dicapryl ether,

synthetic esters, such as the oils of formula R₁COOR₂, in which R₁ represents a linear or branched fatty acid residue comprising from 1 to 40 carbon atoms and R₂ represents a hydrocarbon-based chain, which is especially branched, containing from 1 to 40 carbon atoms, on condition that R₁+R₂≧10. The esters may be chosen especially from fatty acid alcohol esters, for instance cetostearyl octanoate, isopropyl alcohol esters such as isopropyl myristate or isopropyl palmitate, ethyl palmitate, 2-ethylhexyl palmitate, isopropyl stearate, octyl stearate, hydroxylated esters, such as isostearyl lactate or octyl hydroxystearate, alkyl or polyalkyl ricinoleates, hexyl laurate, neopentanoic acid esters, such as isodecyl neopentanoate or isotridecyl neopentanoate, and isononanoic acid esters, such as isononyl isononanoate or isotridecyl isononanoate,

polyol esters and pentaerythritol esters, such as dipentaerythrityl tetrahydroxy stearate/tetraisostearate,

fatty alcohols that are liquid at room temperature, with a branched and/or unsaturated carbon-based chain containing from 12 to 26 carbon atoms, for instance 2-octyldodecanol, isostearyl alcohol and oleyl alcohol,

C₁₂-C₂₂ higher fatty acids, such as oleic acid, linoleic acid, linolenic acid, and mixtures thereof,

non-phenyl silicone oils, for instance caprylyl methicone, and

phenyl silicone oils, for instance phenyl trimethicones, phenyl dimethicones, phenyltrimethylsiloxydiphenylsiloxanes, diphenyl dimethicones, dimethicones or phenyl trimethicone with a viscosity of less than or equal to 100 cSt, and trimethyl-pentaphenyl-trisiloxane, and mixtures thereof, and also mixtures of these various oils.

Preferably, the composition according to the invention comprises less than 10% by weight of non-volatile oil, in particular less than 5% by weight and more particularly comprise no non-volatile oil.

As mentioned above, the gelled oily phase according to the invention may have a threshold stress of greater than 1.5 Pa and preferably greater than 10 Pa.

This threshold stress value reflects a gel-type texture of this oily phase.

Dyestuffs

The compositions in accordance with the invention may comprise at least one dyestuff.

This (or these) dyestuff(s) are preferably chosen from pulverulent dyes, liposoluble dyes and water-soluble dyes, and mixtures thereof.

Preferably, the compositions according to the invention comprise at least one pulverulent dyestuff. The pulverulent dyestuffs may be chosen from pigments and nacres, and preferably from pigments.

The pigments may be white or coloured, mineral and/or organic, and coated or uncoated. Among the mineral pigments, mention may be made of metal oxides, in particular titanium dioxide, optionally surface-treated, zirconium, zinc or cerium oxide, and also iron, titanium or chromium oxide, manganese violet, ultramarine blue, chromium hydrate and ferric blue. Among the organic pigments that may be mentioned are carbon black, pigments of D & C type and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica especially with ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, (3-carotene, soybean oil, Sudan Brown, D&C Yellow 11, D&C Violet 2, D&C Orange 5, quinoline yellow and annatto.

Preferably, the pigments contained in the compositions according to the invention are chosen from metal oxides.

These dyestuffs may be present in a content ranging from 0.01% to 30% by weight, and in particular from 3% to 22% by weight, relative to the total weight of the composition.

Preferably, the dyestuff(s) are chosen from one or more metal oxides that are present in a content of greater than or equal to 2% by weight relative to the total weight of the composition, and advantageously inclusively between 3% and 22% by weight relative to the total weight of the composition.

Fibres

A composition according to the invention, especially when it is intended to be applied to the eyelashes, may also comprise at least one fibre.

The term “fibre” should be understood as meaning an object of length L and of diameter D such that L is greater than D and preferably very much greater than D, D being the diameter of the circle in which the cross section of the fibre is inscribed. In particular, the ratio L/D (or aspect ratio) is chosen in the range from 3.5 to 2500, in particular from 5 to 500 and more particularly from 5 to 150.

The fibres that may be used in the composition of the invention may be mineral or organic fibres, of synthetic or natural origin. They may be short or long, individual or organized, for example braided, and hollow or solid. They may have any shape and may especially have a circular or polygonal (square, hexagonal or octagonal) cross section depending on the specific application envisaged. In particular, their ends are blunted and/or polished to prevent injury.

In particular, the fibres have a length ranging from 1 μm to 10 mm, preferably from 0.1 mm to 5 mm and better still from 0.3 mm to 3 mm. Their cross section may be included in a circle with a diameter ranging from 2 nm to 500 μm, preferably ranging from 100 nm to 100 μm and better still from 1 μm to 50 μm. The weight or yarn count of fibres is often given in denier or decitex and represents the weight in grams per 9 km of yarn. Preferably, the fibres according to the invention have a yarn count chosen within the range from 0.01 to 10 denier, preferably from 0.1 to 2 denier and better still from 0.3/0.7 denier.

The fibres that may be used in the composition of the invention may be chosen from rigid or non-rigid fibres, and may be mineral or organic fibres, of synthetic or natural origin.

Moreover, the fibres may or may not be surface-treated, may be coated or uncoated, and may be coloured or uncoloured.

As fibres that may be used in the composition according to the invention, mention may be made of non-rigid fibres such as polyamide (Nylon®) fibres or rigid fibres such as polyimideamide fibres, for instance those sold under the names Kermel® and Kermel Tech® by the company Rhodia or poly(p-phenyleneterephthalamide) (or aramid) fibres sold especially under the name Kevlar® by the company DuPont de Nemours.

The fibres may be present in a content ranging from 0.01% to 10% by weight, in particular from 0.1% to 5% by weight and more particularly from 0.3% to 3% by weight relative to the total weight of the composition.

Fillers

The compositions in accordance with the invention may also comprise at least one filler.

The fillers may be selected from those that are well known to those skilled in the art and commonly used in cosmetic compositions. The fillers may be mineral or organic, and lamellar or spherical. Mention may be made of mica, talc, silica, kaolin, polyamide powders, for instance the Nylon® sold under the name Orgasol® by the company Atochem, poly-β-alanine powders and polyethylene powders, powders of tetrafluoro-ethylene polymers, for instance Teflon®, lauroyllysine, starch, boron nitride, expanded polymeric hollow microspheres such as those of polyvinylidene chloride/acrylonitrile, for instance the products sold under the name Expancel® by the company Nobel Industrie, acrylic powders such as those sold under the name Polytrap® by the company Dow Corning, polymethyl methacrylate particles and silicone resin microbeads (for example Tospearls® from Toshiba), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids having from 8 to 22 carbon atoms and in particular from 12 to 18 carbon atoms, for example zinc, magnesium or lithium stearate, zinc laurate and magnesium myri state.

The fillers may represent from 0.1% to 15% by weight and in particular from 0.5% to 10% by weight relative to the total weight of the composition.

According to one embodiment of the invention, a composition may comprise at least solid particles such as pigments and/or fillers.

It is a matter of routine operations for a person skilled in the art to adjust the nature and the amount of the additives present in the compositions in accordance with the invention such that the desired cosmetic properties thereof are not thereby affected.

According to a preferred embodiment, a composition of the invention is in the form of a product for the eyelashes, in particular a mascara.

According to another embodiment, a composition of the invention may advantageously be in the form of a product for the eyebrows, in particular an eyebrow pencil.

Preferably, a composition according to the invention is in the form of a composition for caring for and/or making up keratin fibres, in particular the eyelashes, preferably in the form of a mascara.

Such compositions are especially prepared according to the general knowledge of a person skilled in the art.

Throughout the description, including the claims, the term “comprising a” should be understood as being synonymous with “comprising at least one”, unless otherwise specified.

The terms “between . . . and . . . ” and “ranging from . . . to . . . ” should be understood as being inclusive of the limits, unless otherwise specified.

The invention is illustrated in greater detail by the example presented below. Unless otherwise mentioned, the amounts indicated are expressed as mass percentages.

Methodology for the Oscillating Dynamic Rheology Measurements

These are harmonic-regime rheology measurements for measuring the elastic modulus.

The measurements are taken using a Haake RS600 rheometer on a product at rest, at 25° C. with a plate-plate rotor Ø 60 mm and a 2 mm gap.

The harmonic-regime measurements make it possible to characterize the viscoelastic properties of the products. The technique consists in subjecting a material to a stress which varies sinusoidally over time and in measuring the response of the material to this stress. In a range in which the behaviour is linear viscoelastic behaviour (zone in which the strain is proportional to the stress), the stress (τ) and the strain (γ) are two sinusoidal functions of time which are written in the following manner:

τ(t)=τ₀ sin(ωt)

γ(t)=γ₀ sin(ωt+δ)

in which:

τ₀ represents the maximum amplitude of the stress (Pa);

γ₀ represents the maximum amplitude of the strain (−);

ω=2ΠN represents the angular frequency (rad·s⁻¹) with N representing the frequency (Hz); and

δ represents the phase shift of the stress relative to the strain (rad).

Thus, the two functions have the same angular frequency, but they are shifted by an angle δ. Depending on the phase shift δbetween τ(t) and γ(t), the behaviour of the system may be apprehended:

if δ=0, the material is purely elastic;

if δ=Π/2, the material is purely viscous (Newtonian fluid); and

if 0<δ<Π/2, the material is viscoelastic.

In general, the stress and the strain are written in complex form:

τ*(t)=τ₀e^{iωt}

γ*(t)=γ₀e^{(iωt+δ)}

A complex stiffness modulus, representing the overall resistance of the material to the strain, whether it is of elastic or viscous origin, is then defined by:

G*=τ*/γ*=G′+iG″

in which:

G′ is the storage modulus or elastic modulus, which characterizes the energy stored and totally restituted during a cycle, G′=(τ₀/γ₀)cos δ; and

G″ is the loss modulus or viscous modulus, which characterizes the energy dissipated by internal friction during a cycle, G″=(τ₀/γ₀)sin δ.

The parameter retained is the mean stiffness modulus G* recorded at the plateau measured at a frequency of 1 Hz.

### EXAMPLE

### Mascaras

Mascara formulations in accordance with the invention (formulations 1 to 3) or not in accordance with the invention (formulations 4 and 5) are prepared as described below.

To prepare phase A, the hydrophilic gelling agent is added to water in a heating pan with stirring at 70° C. until a homogeneous mixture is obtained. Stirring is adjusted so as not to incorporate air into the mixture.

The rest of the ingredients of phase A, the phenoxyethanol, the pentylene glycol and the denatured alcohol are then added at room temperature.

The components of phase B are weighed out in a heating pan and stirred using a Rayneri blender at 90-95° C.

Once the gels have been prepared and are homogeneous, the two phases are mixed together using a Rayneri blender at room temperature (25° C.).

For the formulations according to the invention, a black homogeneous composition forms.

The formulation is prepared using the weight proportions described below. The percentages are on a weight basis relative to the total weight of the composition.

Smectite is used in the form of a gel in isododecane (Bentone Gel ISD V® sold by the company Elementis) comprising 10% by weight of smectite and 3% by weight of propylene carbonate.

| | | | | | Formulation 4 | Formulation 5 |
|---|---|---|---|---|---|---|
| | | Formulation 1 | Formulation 2 | Formulation 3 | not | not |
| | | according | according | according | according | according |
| | | to | to | to | to | to |
| | | the | the | the | the | the |
| Phase | Compounds | invention | invention | invention | invention | invention |
| Phase A | Steareth-100/PEG 136/HDI | 3.00% | | 3.00% | 3.00% | |
| | (hexamethyl diisocyanate) | | | | | |
| | copolymer (Rheolate ® FX | | | | | |
| | 1100 sold by the company | | | | | |
| | Elementis) | | | | | |
| | Ammonium | | 0.30% | | | |
| | polyacryloyldimethyltauramide | | | | | |
| | (Hostacerin AMPS ® | | | | | |
| | sold by the company | | | | | |
| | Clariant) | | | | | |
| | Pentylene glycol (616751 | 3.00% | 0.15% | 3.00% | 3.00% | 3.00% |
| | Hydrolite ®-5 sold by the | | | | | |
| | company Symrise) | | | | | |
| | Denatured alcohol (Ethanol | 0.90% | 0.90% | 0.90% | 0.90% | 0.90% |
| | SDA 40B 200 proof sold by | | | | | |
| | the company Sasol) | | | | | |
| | Phenoxyethanol (Sepicide | 0.50% | 0.90% | 0.50% | 0.50% | 0.50% |
| | LD sold by the company | | | | | |
| | SEPPIC) | | | | | |
| | Microbiologically clean | 22.60% | 27.75% | 22.60% | 22.60% | 25.60% |
| | deionized water | | | | | |
| Phase B | Hydrogenated | 5.18% | 5.18% | | | 5.18% |
| | styrene/isoprene copolymer | | | | | |
| | (Kraton ® G1701 EU sold | | | | | |
| | by the company Kraton | | | | | |
| | Polymers) | | | | | |
| | Hydrogenated | 10.36% | 10.36% | 42% | 10.36% | 10.36% |
| | styrene/methylstyrene/ | | | | | |
| | indene copolymer | | | | | |
| | (Regalite ® R1100 CG | | | | | |
| | Hydrocarbon Resin sold by | | | | | |
| | the company Eastman | | | | | |
| | Chemical) | | | | | |
| | Iron oxides (Sunpuro black | 4.90% | 4.90% | 5.00%* | 4.90% | 4.90% |
| | iron oxide C33-7001 sold | | | | | |
| | by the company Sun) | | | | | |
| | Phenoxyethanol (Sepicide | 0.35% | 0.35% | | 0.35% | 0.35% |
| | LD sold by the company | | | | | |
| | SEPPIC) | | | | | |
| | Modified smectite | | | 5.00%* | | |
| | (magnesium silicate) in | | | | | |
| | isododecane (Bentone Gel | | | | | |
| | ISD V ® sold by the | | | | | |
| | company Elementis) | | | | | |
| | Isododecane sold by the | 49.21% | 49.21% | 18.00% | 54.39% | 49.21% |
| | company Ineos | | | | | |

*expressed as a weight percentage of commercial product (smectite content: 0.5% by weight)

The textures of the formulations obtained are evaluated macroscopically and microscopically with a Leica DMLB microscope and a Leica ×10 objective lens.

Formulations 1 to 3 (in accordance with the invention) form a macroscopically homogeneous mixture in which the observation by microscope reveals that the oily phase and the aqueous phase are both homogeneous.

Formulations 4 and 5 (comparative), for their part, are in the form of a liquor that is not homogeneous from a macroscopic viewpoint, composed of two immiscible phases, and are consequently not manipulable.

The viscosity of formulations 1, 2 and 3 was measured using a viscometer (Rheomat RM100 from Lamy Rheology, spindle 4). They have viscosities of 9.9, 4.3 and 1.8 Pa·s, respectively.

Viscosities of the comparative formulations could not be evaluated since they do not form a macroscopically homogeneous mixture.

Formulations 1 to 3 obtained are spread on a glass plate to a controlled thickness of 300 μm. After drying for 5 minutes, the tack is evaluated by touching. The formulations according to the invention show a marked decrease in tacky feel, in contrast with the fatty phase alone.

1-26. (canceled)

27. Composition, comprising:
at least one aqueous phase gelled with at least one hydrophilic gelling agent chosen from synthetic polymeric gelling agents, mixed silicates, and mixtures thereof; and
at least one oily phase gelled with at least one lipophilic gelling agent chosen from polymeric gelling agents, particulate gelling agents, and mixtures thereof, said oily phase also comprising at least one volatile oil;
said phases forming therein a macroscopically homogeneous mixture;
said composition also comprising at least one tackifying resin,
said composition comprising a water content at least equal to 15% by weight, relative to the total weight of the composition
said composition comprising from 10% to 70% by weight of volatile oil(s) relative to the total weight of said composition.

28. Composition according to claim 27, comprising from 1% to 60% by weight of tackifying resin(s), relative to the total weight of the composition.

29. Composition according to claim 27, in which said tackifying resin(s) are present totally or partly, in the gelled oily phase.

30. Composition according to claim 27, in which said tackifying resin(s) are chosen from hydrocarbon-based resins.

31. Composition according to claim 27, in which said tackifying resin(s) are chosen from indene/methylstyrene/hydrogenated styrene copolymers.

32. Composition according to claim 27, comprising as lipophilic gelling agent at least one gelling agent chosen from hydrocarbon-based block copolymers, polymers containing hydrogen bonding.

33. Composition according to claim 27, comprising as lipophilic gelling agent at least one gelling agent chosen from copolymers containing styrene blocks and ethylene/C₃-C₄ alkylene blocks, which are hydrogenated; hydrocarbon-based polyamides; bentonites, hectorites; and mixtures thereof.

34. Composition according to claim 27, wherein the gelled oily phase also comprises at least one hydrophobic film-forming polymer.

35. Composition according to claim 34, wherein said hydrophobic film-forming polymer(s) being chosen from lipodispersible film-forming polymers in the form of non-aqueous dispersions of polymer particles, block ethylenic copolymers, vinyl polymers comprising at least one carbosiloxane dendrimer-based unit, silicone acrylate copolymers, and mixtures thereof.

36. Composition according to claim 34, comprising from 1% to 30% by weight of hydrophobic film-forming polymer(s), relative to the total weight of the composition.

37. Composition according to claim 27, comprising, as hydrophilic gelling agent, at least one synthetic polymeric gelling agent.

38. Composition according to claim 27, comprising as hydrophilic gelling agent at least one gelling agent chosen from associative polymers which are nonionic; 2-acrylamido-2-methylpropanesulfonic acid copolymers; and mixtures thereof.

39. Composition according to claim 27, comprising as hydrophilic gelling agent at least one gelling agent chosen from copolymers of 2-acrylamido-2-methylpropanesulfonic acid and of hydroxyethyl acrylate; ammonium 2-acrylamido-2-methylpropanesulfonate polymers; nonionic associative polyurethanes and mixtures thereof.

40. Composition according to claim 27, comprising, as hydrophilic gelling agent/lipophilic gelling agent system, a system chosen from:
nonionic associative polyurethane(s)/hydrocarbon-based block copolymer(s);
nonionic associative polyurethane(s)/polymer(s) containing hydrogen bonding;
nonionic associative polyurethane(s)/modified clays(s);
copolymer(s) of 2-acrylamido-2-methylpropanesulfonic acid and of hydroxyethyl acrylate/hydrocarbon-based block copolymer(s);
copolymer(s) of 2-acrylamido-2-methylpropanesulfonic acid and of hydroxyethyl acrylate/polymer(s) containing hydrogen bonding;
copolymer(s) of 2-acrylamido-2-methylpropanesulfonic acid and of hydroxyethyl acrylate/modified clay(s);
polymer(s) of ammonium 2-acrylamido-2-methylpropanesulfonate/hydrocarbon-based block copolymer(s);
polymer(s) of ammonium 2-acrylamido-2-methylpropanesulfonate/polymer(s) with hydrogen bonding; and
polymer(s) of ammonium 2-acrylamido-2-methylpropanesulfonate/modified clay(s).

41. Composition according to claim 27, containing the aqueous and oily phases in an aqueous phase/oily phase weight ratio of from 10/90 to 90/10.

42. Composition according to claim 27, having a viscosity ranging from 0.5 to 50 Pa·s.

43. Composition according to claim 27, also comprising at least solid particles.

44. Composition according to claim 27, comprising a solids content of greater than or equal to 25%.

45. Composition according to claim 27, comprising less than 5% surfactant.

46. Composition according to claim 27, in the form of a composition for caring for and/or making up keratin fibres.

47. Method for preparing a composition, comprising at least one step of mixing:
an aqueous phase gelled with at least one hydrophilic gelling agent chosen from synthetic polymeric gelling agents, mixed silicates, and mixtures thereof; and
at least one oily phase gelled with at least one lipophilic gelling agent chosen from polymeric gelling agents, particulate gelling agents, and mixtures thereof, said oily phase also comprising at least one volatile oil;
under conditions suitable for obtaining a macroscopically homogeneous mixture;
said composition also comprising at least one tackifying resin,
said composition comprising a water content at least equal to 15% by weight relative to the total weight of the composition,
said composition comprising from 10% to 70% by weight of volatile oil(s) relative to the total weight of said composition.

48. Cosmetic method for making up and/or caring for keratin materials, comprising at least one step which consists in applying to said keratin materials a composition as defined according to claim 27.

49. Cosmetic method for making up and/or caring for keratin materials, comprising at least the application to said keratin materials of a macroscopically homogeneous composition obtained by extemporaneous mixing, before application or at the time of application to said keratin materials, of at least one aqueous phase gelled with at least one hydrophilic gelling agent in particular keratin fibres, comprising at least the application to said keratin materials of a macroscopically homogeneous composition obtained by extemporaneous mixing, before application or at the time of application to said keratin materials, of at least one aqueous phase gelled with at least one hydrophilic gelling agent chosen from synthetic polymeric gelling agents, mixed silicates, and mixtures thereof, and at least one oily phase gelled with at least one lipophilic gelling agent chosen from polymeric gelling agents, particulate gelling agents, and mixtures thereof, said oily phase also comprising at least one volatile oil; said composition also comprising at least one tackifying resin,
said composition comprising a water content at least equal to 15% by weight, relative to the total weight of the composition
said composition comprising from 10% to 70% by weight of volatile oil(s) relative to the total weight of said composition.

50. Composition according to claim 29 in which said tackifying resin(s) are present solely in the gelled oily phase.

51. Composition according to claim 35, in which the hydrophobic film-forming polymer(s) are lipodispersible film-forming polymers in the form of non-aqueous dispersions of polymer particles.
